SEQUENCE LISTING (3 sequences)

SEQ ID NO: 1 - DNA, 1594 bp, Homo sapien
aaagggccgtcctggtccagccgttccctgggtgcccgttgccggaactctatcgcttcctgccctgaggaacaaccccatgtgggcaactataggctgctaaggaccatcgggaagggcaacttcgccaaagtcaagctggctcggcatatcctcacgggccgggaggtcgctattaagatcattgataagacccagctgaaccccagtagcttgcagaagctgttcagagaagtccgaattatgaagggactcaaccaccccaacatcgtgaagctttttgaggtgatagagacggagaagacgctatacctggtgatggaatacgctagcgcaggagaagtgtttgactacctcgtgtcgcacggccgcatgaaggagaaggaggctcgagccaagttccggcagatcgtgtcagccgtgcactactgtcatcagaagaacattgtacacagggatctaaaggctgaaaacctgttgctggatgccgaggccaacatcaaaatcgccgacttcggcttcagcaatgagttcacgctgggctccaagctggacaccttctgtgggagccccccatacgccgccccagagctgttccagggcaagaagtatgatgggccagaggtggacatctggagcctgggtgtcatcctgtacacgctggtcagcggctccctgcccttcgatgggcacaacctcaaggagctgcgggagcgagtcctcagaggaaagtaccgggtccccttctacatgtctacagactgcgagagcattctgcggagatttctggtgctgaaccccgcaaaacgctgtactctggagcaaatcatgaaagacaaatggatcaacatcggctatgagggtgaggagctgaagccagacacggagctcaaagaagagcggatgccgggtcggaaagcgagctgcagtgcagtgggcagtggaagtcgaggcttgcccccctccagccccatggtcagcagtgcccacaaccccaataaggcagagatccctgagcggcggaaggacagcactagcacccctaacaacctcccccccagcatgatgacccgaagaaacacctatgtgtgcacagagcgaccaggatctgaacgcccgtccttgttgccaaatggcaaagaaaatagctccggtacctcgcgggtgccccctgcctcgccttccagtcatagcctggctcccccgtcaggcgagcggagccgcctggctcggggctccaccatccgcagcaccttccatgggggccaggtccgagaccggcgggcagggagcgggagtggcgggggtgtgcagaatggacccccagcctcacccacgcttgcccacgaggccgcacccctgccctccgggcggcctcgccccaccaccaacctcttcaccaagctgacctccaaactgacccgaagggtcacagacgaacctgagagaatcgggggacctgaggtcacaagttgccatctaccttgggataaaacggaaaccgcccccaggctgctccgattcccctggagtgtgaagctgaccagctcgcgaccttcctgaggccctgatggctgccctgcgacaggccaca

SEQ ID NO: 2 - protein, 439 aa, Homo sapien
MKGLNHPNIVKLFEVIETEKTLYLVMEYASAGEVFDYLVSHGRMKEKEARAKFRQIVSAVHYCHQKNIVHRDLKAENLLLDAEANIKIADFGFSNEFTLGSKLDTFCGSPPYAAPELFQGKKYDGPEVDIWSLGVILYTLVSGSLPFDGHNLKELRERVLRGKYRVPFYMSTDCESILRRFLVLNPAKRCTLEQIMKDKWINIGYEGEELKPDTELKEERMPGRKASCSAVGSGSRGLPPSSPMVSSAHNPNKAEIPERRKDSTSTPNNLPPSMMTRRNTYVCTERPGSERPSLLPNGKENSSGTSRVPPASPSSHSLAPPSGERSRLARGSTIRSTFHGGQVRDRRAGSGSGGGVQNGPPASPTLAHEAAPLPSGRPRPTTNLFTKLTSKLTRRVTDEPERIGGPEVTSCHLPWDKTETAPRLLRFPWSVKLTSSRPS

SEQ ID NO: 3 - DNA, 1594 bp, Homo sapien; identical to SEQ ID NO: 1
  CDS (244)...(1563)

(19) United States
(12) Patent Application Publication
(10) Pub. No.: US 2003/0108937 A1
(43) Pub. Date: Jun. 12, 2003

(54) Methods and compositions for the diagnosis and treatment of cellular proliferation disorders using 20750

(75) Inventor: Mark Williamson, Saugus, MA (US)

Correspondence Address: Steven A. Bossone, Millennium Pharmaceuticals, Inc., 75 Sidney Street, Cambridge, MA, 02139, US

(73) Assignee: Millennium Pharmaceuticals, Inc.

(21) Appl. No.: 10/284,060

(22) Filed: Oct. 30, 2002

Related U.S. Application Data
(60) Provisional application No. 60/335,006, filed on Oct. 31, 2001.

(51) Int. Cl. A61K 48/00; A61K 38/17; A61K 31/00; C12Q 1/68; G01N 33/567; A61K 39/395

(52) U.S. Cl. 435/6; 435/7.21; 514/44; 514/1; 514/2; 424/146.1

(57) ABSTRACT

The present invention provides methods and compositions for the diagnosis and treatment of cellular proliferation disorders, e.g., cancer, including, but not limited to colon, breast, and lung cancer. The invention further provides methods for identifying a compound capable of treating a cellular proliferation disorder. The invention also provides methods for identifying a compound capable of modulating a cellular proliferation disorder. In addition, the invention provides a method for treating a subject having a cellular proliferation disorder characterized by aberrant 20750 polypeptide activity or aberrant 20750 nucleic acid expression.

This application claims priority to U.S. provisional application number 60/335,006, filed Oct. 31, 2001, the entire contents of which are herein incorporated by reference.

Cancer is the second leading cause of death in the United States, after heart disease (Boring, et al., (1993) CA Cancer J. Clin. 43:7). Cancer is characterized primarily by an increase in the number of abnormal, or neoplastic, cells derived from a normal tissue which proliferate to form a tumor mass, the invasion of adjacent tissues by these neoplastic tumor cells, and the generation of malignant cells which spread via the blood or lymphatic system to regional lymph nodes and to distant sites. The latter progression to malignancy is referred to as metastasis.

Colorectal cancer is among the most common cancers affecting the western world. An estimated 129,400 new cases of colorectal cancer occurred in the United States in 1999 (Rudy, et al. (2000) Am Fam Physician 61(6):1759-70, 1773-4). By the age of 70 years, at least 50% of the Western population will develop some form of colorectal tumor, including early benign polyps and invasive adenocarcinomas. It is estimated that approximately 10% of the benign polypoid lesions will progress to invasive carcinoma (Fahy et al. (1998) Surg Oncol 7(3-4):115-23). Colorectal cancer arises from a precursor lesion, the adenomatous polyp, which forms in a field of epithelial cell hyperproliferation and crypt dysplasia. Progression from this precursor lesion to colorectal cancer is a multistep process (Winawer (1999) Am J Med 106(1A):3S-6S).

Lung cancer is the most common form of cancer in the world. Estimates for the year 1985 indicate that there were about 900,000 cases of lung cancer worldwide. (Parkin et al., (1993) Int J Cancer; 54:594-606). For the United States alone, 1993 projections placed the number of new lung cancer cases at 170,000, with a mortality of about 88%. (Boring et al., supra). Although the occurrence of breast cancer is slightly more common in the United States, lung cancer is second behind prostate cancer for males and third behind breast and colorectal cancers for women. Yet, lung cancer is the most common cause of cancer deaths.

The World Health Organization classifies lung cancer into four major histological types: (1) squamous cell carcinoma (SCC), (2) adenocarcinoma, (3) large cell carcinoma, and (4) small cell lung carcinoma (SCLC). (The World Health Organization, Am J Clin Pathol (1982) 77:123-136). However, there is a great deal of tumor heterogeneity even within the various subtypes, and it is not uncommon for lung cancer to have features of more than one morphologic subtype. The term non-small cell lung carcinoma (NSCLC) includes squamous, adenocarcinoma and large cell carcinomas.

Early detection is difficult since clinical symptoms are often not seen until the disease has reached an advanced stage. Currently, diagnosis is aided by the use of chest x-rays, analysis of the type of cells contained in sputum and fiberoptic examination of the bronchial passages. Treatment regimens are determined by the type and stage of the cancer, and include surgery, radiation therapy and/or chemotherapy. In spite of considerable research into therapies for the disease, lung cancer remains difficult to treat.

Breast carcinoma or cancer is a major medical problem for women beginning in the third decade of life and continuing throughout senescence. It is currently estimated that in the United States women have a one in eight chance of developing the disease in their lifetime (by the age of eighty), whereas one in twenty-eight women have a lifetime risk of dying from breast cancer (Harris et al., Ed. Diseases of the Breast, 1996: pp. 159-168). Carcinoma of the breast is the third most common cancer, and the most common cancer in women. It is a major cause of mortality in women, as well as a cause of disability, psychological trauma, and economic loss. Breast carcinoma is the second most common cause of cancer death in women in the United States, and for women between the ages of 15 and 54, the leading cause of cancer-related death (Forbes, Seminars in Oncology, vol. 24(1), Suppl 1, 1997: pp.S1-20-S1-35). Indirect effects of the disease also contribute to the mortality from breast cancer including consequences of advanced disease, such as metastases to the bone or brain. Complications arising from bone marrow suppression, radiation fibrosis and neutropenic sepsis, collateral effects from therapeutic interventions, such as surgery, radiation, chemotherapy, or bone marrow transplantation-also contribute to the morbidity and mortality from this disease.

Based on the prevalence of these disorders and the lack of effective cures and early diagnostics, there currently exists a great need for methods and compositions which can serve as markers before the onset of symptoms and which can serve as a means for identifying therapeutics to treat and or cure these disorders.

The present invention provides methods and compositions for the diagnosis and treatment of cellular proliferation disorders, e.g., cancer, including, but not limited to colon cancer, breast cancer, and lung cancer. The present invention is based, at least in part, on the discovery that the 20750 molecule, a novel member of the protein kinase family, is differentially expressed in tumor cells, e.g., colon, lung, and breast tumor cells, as compared to normal cells, e.g., normal colon, lung, and breast cells, and is highly elevated in tissues derived from colon metastases to the liver as compared to normal liver tissue, and thus is useful in the diagnosis and treatment of cellular proliferation disorders, e.g., cancer, including, but not limited to, colon, lung, and breast cancer. Without intending to be limited by mechanism, it is believed that the 20750 molecules, by influencing the stability of β-catenin, modulate carcinogenesis through the Wnt-signaling pathway and are, therefore, useful as targets and therapeutic agents for the treatment, prognosis, or diagnosis, of cellular proliferation disorders, such as cancer.

Accordingly, the present invention provides methods for the diagnosis and treatment of cellular proliferation disorders, e.g., cancer, including, but not limited to, colon, breast, and lung cancer.

In one aspect, the invention provides methods for identifying a compound capable of treating a cellular proliferation disorder, e.g., cancer, including, but not limited to colon, breast, and lung cancer. The method includes assaying the ability of the compound to modulate 20750 nucleic acid expression or 20750 polypeptide activity. In one embodiment, the ability of the compound to modulate nucleic acid expression or 20750 polypeptide activity is determined by detecting modulation of cellular proliferation, e.g., proliferation of a tumor cell. In another embodiment, the ability of the compound to modulate nucleic acid expression or 20750 polypeptide activity is determined by detecting modulation of the concentration of β-catenin in a cell.

In another aspect, the invention provides methods for identifying a compound capable of modulating a 20750 activity, e.g., intracellular β-catenin concentration, cellular proliferation, cellular growth, cellular migration or the Wnt-signaling pathway. The method includes contacting a cell expressing a 20750 nucleic acid or polypeptide molecule (e.g., a colon tumor cell, a lung tumor cell, or a breast tumor cell) with a test compound and assaying the ability of the test compound to modulate 20750 nucleic acid expression or 20750 polypeptide activity.

Another aspect of the invention provides a method for modulating a cellular growth, migration differentiation, or proliferation process. The method includes contacting a cell with a 20750 modulator, for example, an anti-20750 antibody, a 20750 polypeptide comprising the amino acid sequence of SEQ ID NO:2 or a fragment thereof, a 20750 polypeptide comprising an amino acid sequence which is at least 90 percent identical to the amino acid sequence of SEQ ID NO:2, an isolated naturally occurring allelic variant of a polypeptide consisting of the amino acid sequence of SEQ ID NO:2, a small molecule, an antisense 20750 nucleic acid molecule, a nucleic acid molecule of SEQ ID NO:1 or a fragment thereof, or a ribozyme.

In yet another aspect, the invention features a method for treating a subject having a cellular proliferation disorder, e.g., a cellular proliferation disorder characterized by aberrant 20750 polypeptide activity or aberrant 20750 nucleic acid expression, such as cancer. In a preferred embodiment, the cellular proliferation disorder is colon cancer, lung cancer, or breast cancer. The method includes administering to the subject a therapeutically effective amount of a 20750 modulator, e.g., in a pharmaceutically acceptable formulation or by using a gene therapy vector. Embodiments of this aspect of the invention include the 20750 modulator being a small molecule, an anti-20750 antibody, a 20750 polypeptide comprising the amino acid sequence of SEQ ID NO:2 or a fragment thereof, a 20750 polypeptide comprising an amino acid sequence which is at least 90 percent identical to the amino acid sequence of SEQ ID NO:2, an isolated naturally occurring allelic variant of a polypeptide consisting of the amino acid sequence of SEQ ID NO:2, an antisense 20750 nucleic acid molecule, a nucleic acid molecule of SEQ ID NO: 1 or a fragment thereof, or a ribozyme.

In another aspect, the invention provides a method for modulating, e.g., increasing or decreasing, cellular proliferation in a subject by administering to the subject a 20750 modulator.

Also featured are methods of regulating metastasis in a subject or inhibiting tumor progression in a subject which include administering to the subject an effective amount of an 20750 modulator (e.g., an 20750 inhibitor).

The present invention is based also in part, on the discovery of novel members of the family of protein kinases, referred to herein as “20750” nucleic acid and protein molecules. The 20750 nucleic acid and protein molecules of the present invention are useful as modulating agents in regulating a variety of cellular processes, e.g., β-catenin stability, cellular proliferation, cellular growth, cellular migration, or the Wnt-signaling pathway.

In one embodiment, the invention features an isolated nucleic acid molecule that includes the nucleotide sequence set forth in SEQ ID NO:1 or SEQ ID NO:3. In another embodiment, the invention features an isolated nucleic acid molecule that encodes a polypeptide including the amino acid sequence set forth in SEQ ID NO:2.

In still other embodiments, the invention features isolated nucleic acid molecules including nucleotide sequences that are substantially identical (e.g., 60% identical) to the nucleotide sequence set forth as SEQ ID NO:1 or SEQ ID NO:3. The invention further features isolated nucleic acid molecules including at least 68 contiguous nucleotides of the nucleotide sequence set forth as SEQ ID NO:1 or SEQ ID NO:3. In another embodiment, the invention features isolated nucleic acid molecules which encode a polypeptide including an amino acid sequence that is substantially identical (e.g., 60% identical) to the amino acid sequence set forth as SEQ ID NO:2. The present invention also features nucleic acid molecules which encode allelic variants of the polypeptide having the amino acid sequence set forth as SEQ ID NO:2. In addition to isolated nucleic acid molecules encoding full-length polypeptides, the present invention also features nucleic acid molecules which encode fragments, for example, biologically active or antigenic fragments, of the full-length polypeptides of the present invention (e.g., fragments including at least 215 contiguous amino acid residues of the amino acid sequence of SEQ ID NO:2). In still other embodiments, the invention features nucleic acid molecules that are complementary to, antisense to, or hybridize under stringent conditions to the isolated nucleic acid molecules described herein.

In another aspect, the invention provides vectors including the isolated nucleic acid molecules described herein (e.g., 20750-encoding nucleic acid molecules). Such vectors can optionally include nucleotide sequences encoding heterologous polypeptides. Also featured are host cells including such vectors (e.g., host cells including vectors suitable for producing 20750 nucleic acid molecules and polypeptides).

In another aspect, the invention features isolated 20750 polypeptides and/or biologically active or antigenic fragments thereof. Exemplary embodiments feature a polypeptide including the amino acid sequence set forth as SEQ ID NO:2, a polypeptide including an amino acid sequence at least 60% identical to the amino acid sequence set forth as SEQ ID NO:2, a polypeptide encoded by a nucleic acid molecule including a nucleotide sequence at least 60% identical to the nucleotide sequence set forth as SEQ ID NO:1 or SEQ ID NO:3. Also featured are fragments of the full-length polypeptides described herein (e.g., fragments including at least 215 contiguous amino acid residues of the sequence set forth as SEQ ID NO:2) as well as allelic variants of the polypeptide having the amino acid sequence set forth as SEQ ID NO:2.

The 20750 polypeptides and/or biologically active or antigenic fragments thereof, are useful, for example, as reagents or targets in assays applicable to treatment and/or diagnosis of 20750 mediated or related disorders. In one embodiment, a 20750 polypeptide or fragment thereof, has a 20750 activity. In another embodiment, a 20750 polypeptide or fragment thereof, includes at least one of the following domains: a transmembrane domain, a protein kinase domain, and optionally, has a 20750 activity. In a related aspect, the invention features antibodies (e.g., antibodies which specifically bind to any one of the polypeptides described herein) as well as fusion polypeptides including all or a fragment of a polypeptide described herein.

The present invention further features methods for detecting 20750 polypeptides and/or 20750 nucleic acid molecules, such methods featuring, for example, a probe, primer or antibody described herein. Also featured are kits e.g., kits for the detection of 20750 polypeptides and/or 20750 nucleic acid molecules. In a related aspect, the invention features methods for identifying compounds which bind to and/or modulate the activity of a 20750 polypeptide or 20750 nucleic acid molecule described herein. Further featured are methods for modulating a 20750 activity.

Other features and advantages of the invention will be apparent from the following detailed description and claims.

Table 1 is a graph depicting the results of a TaqMan™ analysis of 20750 cDNA expression in a human normal and tumor tissue panel (1=normal artery, 2=diseased artery, 3=normal vein, 4=coronary smooth muscle cells, 5=HUVEC, 6=hemangioma, 7=normal heart tissue, 8=congestive heart failure (CHF) heart tissue, 9=kidney tissue, 10=skeletal muscle, 11=normal adipose, 12=pancreas tissue, 13=primary osteoblasts, 14=differentiated osteoclasts, 15=normal skin tissue, 16=normal spinal cord, 17=normal brain cortex, 18=brain hypothalamus, 19=nerve tissue, 20=dorsal root ganglia (DRG), 21=normal breast tissue, 22=normal ovary tissue, 23=ovary tumor tissue, 24=normal prostate tissue, 25=prostate tumor tissue, 26=salivary glands, 27=normal colon tissue, 28=colon tumor tissue, 29=lung normal tissue, 30=lung tumor tissue, 31=chronic obstructive pulmonary disease (COPD) lung tissue, 32=inflammatory bowel disease (IBD) colon tissue, 33=normal liver tissue, 34=liver fibrosis tissue, 34=normal spleen tissue, 36=normal tonsil tissue, 37=lymph node tissue, 38=small intestine tissue, 39=macrophages, 40=synovium, 41=bone marrow, 42=activated PBMC, 43=neutrophils, 44=megakaryocytes, 45=erythroid tissue, 46=positive control).

Table 2 is a graph depicting the results of a TaqMan™ analysis of 20750 cDNA expression in a xenograft panel comprising breast, colon, lung and breast cancer cell lines as well as 293 and 293T cell lines(1=MCF-7 breast tumor, 2=ZR75 breast tumor, 3=T47D breast tumor, 4=MDA 231 breast tumor, 5=MDA 435 breast tumor, 6=SkBr3 breast tumor, 7=DLD-1 colon tumor (stage C), 8=SW480 colon tumor (stage B), 9=SW620 colon tumor (stage C), 10=HCT-116 colon tumor, 11=HT-29 colon tumor, 12=Colo-205 colon tumor, 13=NCI-H125 lung tumor, 14=NCI-H69 lung tumor, 15=NCI-H322 primary bronchioloalveolar carcinoma, 16=NCI-H460 lung tumor, 17=A549 lung tumor, 18=normal human bronchial epithelium (NHBE), 19=SKOV-3 ovary tumor, 20=OVCAR-3 ovary tumor, 21=293 baby kidney, 22=293T baby kidney).

Table 3 is a graph depicting the results of a TaqMan™ analysis of 20750 cDNA expression in an oncology human panel comprising normal and solid tumor samples (1-3=normal breast, 4-8=breast tumor, 9=lymph, 10=lung (breast), 11-12=normal ovary, 13-17=ovary tumor, 18-20=normal lung, 21-26=lung tumor, 27-29=normal colon, 30-33=colon tumor, 34-35=colon tumor—liver metastasis, 36=normal liver (female), 37=cervix, 38=cervix—squamous, 39=human microvascular endothelial cells (HMVECs), arrested, 40=human microvascular endothelial cells (HmVECs), proliferating, 41=hemangioma, 42=HCT-116 normoxic, 43=HCT-116 hypoxic, 44-45=prostate, 46=normal prostate tumor, 47=prostate tumor).

Table 4 is a graph depicting the results of a TaqMan™ analysis of 20750 cDNA expression in an panel comprising normal colon samples, early stage adenocarcinomas, colon to liver metastases, and normal liver samples (1-3=normal colon, 4-5=colonic ACA-C, 6=colonic ACA-B, 7=adenocarcinoma, 8-24=colon to liver metastases, 25-27=normal liver).

Table 5 is a graph depicting the results of a TaqMan™ analysis of 20750 cDNA expression in a colonic ACA panel (1-5=normal colon samples, 6-7=adenomas, 8-11=stage B adenocarcinoma samples, 12-17=stage C adenocarcinoma samples, 18-22=normal liver samples, 23-27=colon to liver metastasis samples, 28=abdominal colon metastasis).

Table 6 is a graph depicting the results of a TaqMan™ analysis of 20750 cDNA expression in an in vitro synchronized cell cycle panel (1=HCT-116 aphidicolin t=0, 2=HCT-116 aphidicolin t=3, 3=HCT-116 aphidicolin t=6, 4=HCT-116 aphidicolin t=9, 5=HCT-116 aphidicolin t=12, 6=HCT-116 aphidicolin t=15, 7=HCT-116 aphidicolin t=18, 8=HCT-116 aphidicolin t=21, 9=HCT-116 aphidicolin t=24, 10=HCT-116 nocodazole t=0, 11=HCT-116 nocodazole t=3, 12=HCT-116 nocodazole t=6, 13=HCT-116 nocodazole t=9, 14=HCT-116 nocodazole t=15, 15=HCT-116 nocodazole t=18, 16=HCT-116 nocodazole t=21, 17=HCT-116 nocodazole t=24, 18=DLD-1 nocodazole t=3, 19=DLD-1 nocodazole t=6, 20=DLD-1 nocodazole t=9, 21=DLD-1 nocodazole t=12, 22=DLD-1 nocodazole t=15, 23=DLD-1 nocodazole t=18, 24=DLD-1 nocodazole t=21, 25=A549 mimosine t=0, 26=A549 mimosine t=3, 27=A549 mimosine t=6, 28=A549 mimosine t=9, 29=A549 mimosine t=15, 30=A549 mimosine t=18, 31=A549 mimosine t=21, 32=A549 mimosine t=24, 33=MCF10A mimosine t=0, 34=MCF10A mimosine t=3, 35=MCF10A mimosine t=6, 36=MCF10A mimosine t=9, 37=MCF10A mimosine t=12, 38=MCF10A mimosine t=18, 39=MCF10A mimosine t=21, 40=MCF10A mimosine t=24).

Table 7 is a graph depicting the results of a TaqMan™ analysis of 20750 cDNA expression in an in vitro oncogene cell model panel comprising various cell lines transfected with k-ras (1=Smad4-Sw480 control, 2=Smad4-Sw480 (24 hours), 3=Smad4-Sw480 (48 hours), 4=Smad4-Sw480 (72 hours), 5=L51747-mucinous, 6=HT29 non-mucinous, 7=SW620 non-mucinous, 8=CSC-1 normal, 9=NCM-460 normal, 10=HCT-116 rer+, 11=SW480 RER+, 12=SW480 rer −/−, 13=CACO rer −/−, 14=JDLD-1, 15=JHCT-116, 16=DKO1, 17=DKO4, 18=DKS-8, 19=Hke3, 20=HKh2, 21=HK2-6, 22=e3Ham#9, 23=APC5 −/−, 24=AP6 −/−, 25=APC1 +/+, 26=APC13 +/+).

Table 8 is a graph depicting a larger view various samples from the TaqMan™ analysis of 20750 cDNA expression in an in vitro oncogene cell model panel shown in Table 7 (1=JDLD-1, 2=DKO1, 3=DKO4, 4=DKS-8, 5=JHCT-116, 6=HK2-6, 7=Hke3, 8=HKh2, 9=eHam#9).

Table 9 is a graph depicting 20750 expression in a Smad3^{-/-} mouse model. Expression in normal colon samples at (1)12-14 weeks and (2)18-24 weeks and adenoma samples at (3)12-14 weeks and (4)18-24 weeks was investigated.

Table 10 is a graph depicting 20750 expression in cell cycle regulated HCT-116 human colon carcinoma cells treated with nocodazole at various time points (1=t=0 h, 2=t=3 h, 3=t=6 h, 4=t=9 h, 5=t=15 h, 6=t=18 h, 7=t=21 h, 8=t=24 h).

TABLE 1

| Sample | | Relative |
|---|---|---|
| Number | Tissue Type | Expression |
| 1 | Artery normal | 20.0535 |
| 2 | Aorta diseased | 7.239 |
| 3 | Vein normal | 9.8204 |
| 4 | Coronary SMC | 62.5 |
| 5 | HUVEC | 231.647 |
| 6 | Hemangioma | 20.4749 |
| 7 | Heart normal | 39.83 |
| 8 | Heart CHF | 23.8478 |
| 9 | Kidney | 19.1038 |
| 10 | Skeletal Muscle | 42.9857 |
| 11 | Adipose normal | 10.9343 |
| 12 | Pancreas | 27.6802 |
| 13 | primary osteoblasts | 25.9162 |
| 14 | Osteoclasts (diff) | 3.9471 |
| 15 | Skin normal | 32.2401 |
| 16 | Spinal cord normal | 14.5786 |
| 17 | Brain Cortex normal | 177.3904 |
| 18 | Brain Hypothalamus normal | 70.0729 |
| 19 | Nerve | 15.4634 |
| 20 | DRG (Dorsal Root Ganglion) | 33.4929 |
| 21 | Breast normal | 19.6408 |
| 22 | Ovary normal | 14.6293 |
| 23 | Ovary Tumor | 11.3592 |
| 24 | Prostate Normal | 16.8046 |
| 25 | Prostate Tumor | 29.1573 |
| 26 | Salivary glands | 9.3553 |
| 27 | Colon normal | 6.6382 |
| 28 | Colon Tumor | 16.8629 |
| 29 | Lung normal | 8.7591 |
| 30 | Lung tumor | 29.7699 |
| 31 | Lung COPD | 6.1084 |
| 32 | Colon IBD | 1.4347 |
| 33 | Liver normal | 4.996 |
| 34 | Liver fibrosis | 19.1701 |
| 35 | Spleen normal | 6.1722 |
| 36 | Tonsil normal | 8.9742 |
| 37 | Lymph node normal | 12.0904 |
| 38 | Small intestine normal | 4.0022 |
| 39 | Macrophages | 2.5329 |
| 40 | Synovium | 1.835 |
| 41 | BM-MNC | 5.4294 |
| 42 | Activated PBMC | 2.6588 |
| 43 | Neutrophils | 3.4962 |
| 44 | Megakaryocytes | 20.0535 |
| 45 | Erythroid | 25.3829 |
| 46 | positive control | 108.8188 |

TABLE 2

| Sample | | Relative |
|---|---|---|
| Number | Tissue Type | Expression |
| 1 | MCF-7 Breast T | 61.0 |
| 2 | ZR75 Breast T | 290.2 |
| 3 | T47D Breast T | 55.0 |
| 4 | MDA 231 Breast T | 45.6 |
| 5 | MDA 435 Breast T | 20.2 |
| 6 | SKBr3 Breast | 67.7 |
| 7 | DLD 1 ColonT (stageC) | 258.8 |
| 8 | SW480 Colon T (stage B) | 44.2 |
| 9 | SW620 ColonT (stageC) | 45.3 |
| 10 | HCT116 | 119.1 |
| 11 | HT29 | 21.3 |
| 12 | Colo 205 | 7.8 |
| 13 | NCIH125 | 244.9 |
| 14 | NCIH67 | 143.6 |
| 15 | NCIH322 | 119.9 |
| 16 | NCIH460 | 116.2 |
| 17 | A549 | 113.4 |
| 18 | NHBE | 288.2 |
| 19 | SKOV-3 ovary | 58.1 |
| 20 | OVCAR-3 ovary | 75.9 |
| 21 | 293 Baby Kidney | 390.9 |
| 22 | 293T Baby Kidney | 277.4 |

TABLE 3

| Sample | | Relative |
|---|---|---|
| Number | Tissue Type | Expression |
| 1 | PIT 400 Breast N | 1.95 |
| 2 | PIT 372 Breast N | 3.33 |
| 3 | CHT 1228 Breast N | 19.92 |
| 4 | MDA 304 Breast T: | 9.49 |
| | MD-IDC | |
| 5 | CHT 2002 Breast T: IDC | 20.69 |
| 6 | MDA 236-Breast T: | 5.45 |
| | PD-IDC(ILC?) | |
| 7 | CHT 562 Breast T: IDC | 10.27 |
| 8 | NDR 138 Breast T ILC | 31.91 |
| | (LG) | |
| 9 | CHT 1841 Lymph node | 25.38 |
| | (Breast met) | |
| 10 | PIT 58 Lung (Breast met) | 11.28 |
| 11 | CHT 620 Ovary N | 27.39 |
| 12 | CHT 619 Ovary N | 61.21 |
| 13 | CLN 012 Ovary T | 22.17 |
| 14 | CLN 07 Ovary T | 4.76 |
| 15 | CLN 17 Ovary T | 14.48 |
| 16 | MDA 25 Ovary T | 12.96 |
| 17 | CLN 08 Ovary T | 5.10 |
| 18 | PIT 298 Lung N | 7.95 |
| 19 | MDA 185 Lung N | 1.65 |
| 20 | CLN 930 Lung N | 4.06 |
| 21 | MPI 215 Lung T-SmC | 37.68 |
| 22 | MDA 259 Lung T-PDNSCCL | 43.59 |
| 23 | CHT 832 Lung T- | 14.99 |
| | PDNSCCL | |
| 24 | MDA 262 Lung T-SCC | 37.16 |
| 25 | CHT 793 Lung T-ACA | 25.65 |
| 26 | CHT 331 Lung T-ACA | 33.49 |
| 27 | CHT 405 Colon N | 1.63 |
| 28 | CHT 1685 Colon N | 13.65 |
| 29 | CHT 371 Colon N | 2.14 |
| 30 | CHT 382 Colon T: MD | 2.70 |
| 31 | CHT 528 Colon T: MD | 6.90 |
| 32 | CLN 609 Colon T | 13.37 |
| 33 | NDR 210 Colon T: MD-PD | 8.58 |
| 34 | CHT 340 Colon-Liver Met | 20.98 |
| 35 | CHT 1637 Colon-Liver Met | 14.58 |
| 36 | PIT 260 Liver N (female) | 2.66 |
| 37 | CHT 1653 Cervix Squamous | 43.28 |
| | CC | |
| 38 | CHT 569 Cervix Squamous | 7.63 |
| | CC | |
| 39 | A24 HMVEC-Arr | 83.33 |
| 40 | C48 HMVEC-Prol | 60.58 |
| 41 | Pooled Hemangiomas | 5.60 |
| 42 | HCT116N22 Normoxic | 241.48 |
| 43 | HCT116H22 Hypoxic | 190.78 |
| 44 | CHT 31 Prostate N | 27.11 |
| 45 | CHT 33 Prostate N | 15.46 |
| 46 | CHT 1269 Prostate T: St 5 | 37.42 |
| 47 | PIT 120 Prostate T: St 7 | 43.28 |

TABLE 4

| | Relative |
|---|---|
| Tissue Type | Expression |
| CHT 371 Colon N | 1.04 |
| CHT 523 Colon N | 9.62 |
| NDR 104 Colon N | 11.72 |
| CHT 520 Colonic ACA-C | 7.21 |
| CHT 1365 Colonic ACA-C | 11.01 |
| CHT 382 Colonic ACA-B | 8.70 |
| CHT 122 Adenocarcinoma | 16.98 |
| CHT 077 Liver-Colon Mets | 11.88 |
| CHT 739 Liver-Colon Mets | 12.26 |
| CHT 755 Liver-Colon Mets | 21.94 |
| CHT001 Liver-Colon Mets | 11.64 |
| CHT 084 Liver-Colon Mets | 25.38 |
| CHT 113 Liver-Colon Mets | 4.73 |
| CHT 114 Liver-Colon Mets | 25.38 |
| CHT 127 Liver-Colon Mets | 29.36 |
| CHT 137 Liver-Colon Mets | 22.80 |
| CHT 218 Liver-Colon Mets | 14.89 |
| CHT 220 Liver-Colon Mets | 15.04 |
| CHT 324 Liver-Colon Mets | 14.03 |
| CHT 340 Liver-Colon Met | 17.28 |
| CHT 530 Liver-Colon Met | 13.56 |
| CHT 849 Liver-Colon Met | 10.78 |
| CHT 1637 Liver-Colon Met | 12.01 |
| CHT131 Liver-Colon Met | 15.04 |
| NDR 165 Liver Normal | 7.52 |
| NDR 150 Liver Normal | 6.39 |
| PIT 236 Liver Normal | 3.25 |

TABLE 5

| Sample | | Relative |
|---|---|---|
| Number | Tissue Type | Expression |
| 1 | CHT 410 Colon N | 1.51 |
| 2 | CHT 425 Colon N | 5.74 |
| 3 | CHT 371 Colon N | 1.03 |
| 4 | PIT 281 Colon N | 45.44 |
| 5 | NDR 211 Colon N | 4.73 |
| 6 | CHT 122 Adenomas | 17.10 |
| 7 | CHT 887 Adenomas | 33.38 |
| 8 | CHT 414 Colonic ACA-B | 16.92 |
| 9 | CHT 841 Colonic ACA-B | 20.62 |
| 10 | CHT 890 Colonic ACA-B | 4.20 |
| 11 | CHT 377 Colonic ACA-B | 5.35 |
| 12 | CHT 520 Colonic ACA-C | 4.74 |
| 13 | CHT 596 Colonic ACA-C | 1.16 |
| 14 | CHT 907 Colonic ACA-C | 6.02 |
| 15 | CHT 372 Colonic ACA-C | 9.99 |
| 16 | NDR 210 Colonic ACA-C | 5.98 |
| 17 | CHT 1365 Colonic ACA-C | 5.35 |
| 18 | CLN 740 Liver N | 7.81 |
| 19 | CLN 741 Liver N | 13.79 |
| 20 | NDR 165 Liver N | 6.48 |
| 21 | NDR 150 Liver N | 10.34 |
| 22 | CHT 1878 Liver N | 9.79 |
| 23 | CHT 119 Col Liver Met | 26.10 |
| 24 | CHT 131 Col Liver Met | 13.79 |
| 25 | CHT 218 Col Liver Met | 14.38 |
| 26 | CHT 739 Col Liver Met | 11.84 |
| 27 | CHT 755 Col Liver Met | 10.78 |
| 28 | CHT 215 Col Abdominal Met | 1.12 |

TABLE 6

| Sample | | Relative |
|---|---|---|
| Number | Tissue Type | Expression |
| 1 | HCT 116 Aphidl t = 0 | 76 |
| 2 | HCT 116 Aphidl t = 3 | 86 |
| 3 | HCT 116 Aphidl t = 6 | 62 |
| 4 | HCT 116 Aphidl t = 9 | 126 |
| 5 | HCT 116 Aphidl t = 12 | 83 |
| 6 | HCT 116 Aphidl t = 15 | 80 |
| 7 | HCT 116 Aphidl t = 18 | 74 |
| 8 | HCT 116 Aphidl t = 21 | 92 |
| 9 | HCT 116 Aphidl t = 24 | 81 |
| 10 | HCT 116 Noc t = 0 | 149 |
| 11 | HCT 116 Noc t = 3 | 133 |
| 12 | HCT 116 Noc t = 6 | 130 |
| 13 | HCT 116 Noc t = 9 | 68 |
| 14 | HCT 116 Noc t = 15 | 113 |
| 15 | HCT 116 Noc t = 18 | 114 |
| 16 | HCT 116 Noc t = 21 | 153 |
| 17 | HCT 116 Noc t = 24 | 214 |
| 18 | DLD noc t = 3 | 180 |
| 19 | DLD noc t = 6 | 171 |
| 20 | DLD noc t = 9 | 212 |
| 21 | DLD noc t = 12 | 239 |
| 22 | DLD noc t = 15 | 374 |
| 23 | DLD noc t = 18 | 360 |
| 24 | DLD noc t = 21 | 151 |
| 25 | A549 Mimo t = 0 | 93 |
| 26 | A549 Mimo t = 3 | 108 |
| 27 | A549 Mimo t = 6 | 125 |
| 28 | A549 Mimo t = 9 | 147 |
| 29 | A549 Mimo t = 15 | 76 |
| 30 | A549 Mimo t = 18 | 68 |
| 31 | A549 Mimo t = 21 | 73 |
| 32 | A549 Mimo t = 24 | 68 |
| 33 | MCF10A Mimo t = 0 | 135 |
| 34 | MCF10A Mimo t = 3 | 186 |
| 35 | MCF10A Mimo t = 6 | 113 |
| 36 | MCF10A Mimo t = 9 | 84 |
| 37 | MCF10A Mimo t = 12 | 94 |
| 38 | MCF10A Mimo t = 18 | 108 |
| 39 | MCF10A Mimo t = 21 | 128 |
| 40 | MCF10A Mimo t = 24 | 110 |

TABLE 7

| Sample | | Relative |
|---|---|---|
| Number | Tissue Type | Expression |
| 1 | SMAD4-SW480 C | 33.61 |
| 2 | SMAD4-SW480 24 HR | 42.25 |
| 3 | SMAD4-SW480 48 HR | 54.22 |
| 4 | SMAD4-SW480 72 HR | 35.16 |
| 5 | L51747-MUCINOUS | 51.83 |
| 6 | HT29 NON-MUCINOUS | 45.75 |
| 7 | SW620 NON-MUCINOUS | 34.67 |
| 8 | CSC-1 NORMAL | 57.91 |
| 9 | NCM-460 NORMAL | 61.64 |
| 10 | HCT116 RER+ | 76.68 |
| 11 | SW48 RER+ | 303.55 |
| 12 | SW480 RER−/− | 54.22 |
| 13 | CACO- RER−/− | 48.03 |
| 14 | JDLD-1 | 227.67 |
| 15 | JHCT116 | 123.28 |
| 16 | DKO1 | 197.51 |
| 17 | DKO4 | 321.97 |
| 18 | DKS-8 | 275.48 |
| 19 | HKe3 | 96.72 |
| 20 | HKh2 | 79.66 |
| 21 | HK2-6 | 175.56 |
| 22 | e3Ham#9 | 73.05 |
| 23 | APC5−/− | 0.00 |
| 24 | APC6−/− | 2.00 |
| 25 | APC1+/+ | 0.31 |
| 26 | APC13+/+ | 0.61 |

TABLE 8

| Sample | | Relative |
|---|---|---|
| Number | Tissue Type | Expression |
| 1 | JDLD-1 | 227.67 |
| 2 | DKO1 | 197.51 |
| 3 | DKO4 | 321.97 |
| 4 | DKS-8 | 275.48 |
| 5 | JHCT116 | 123.28 |
| 6 | HK2-6 | 175.56 |
| 7 | HKe3 | 96.72 |
| 8 | HKh2 | 79.66 |
| 9 | e3Ham#9 | 73.05 |

TABLE 9

| Sample | | Relative |
|---|---|---|
| Number | Tissue Type | Expression |
| 1 | Normal colon 12-14 weeks | 3 |
| 2 | Normal colon 18-24 weeks | 2.9 |
| 3 | Adenoma 12-14 weeks | 5.9 |
| 4 | Adenoma 18-24 weeks | 5.4 |

TABLE 10

| Sample | | Relative |
|---|---|---|
| Number | Tissue Type | Expression |
| 1 | HCT 116 Noc t = 0 | 149 |
| 2 | HCT 116 Noc t = 3 | 133 |
| 3 | HCT 116 Noc t = 6 | 130 |
| 4 | HCT 116 Noc t = 9 | 68 |
| 6 | HCT 116 Noc t = 15 | 113 |
| 7 | HCT 116 Noc t = 18 | 114 |
| 8 | HCT 116 Noc t = 21 | 153 |
| 9 | HCT 116 Noc t = 24 | 214 |

The present invention provides methods and compositions for the diagnosis and treatment of cellular proliferation disorders, e.g., cancer, including, but not limited to, colon, breast, and lung cancer. The present invention is based, at least in part, on the discovery that a novel member of the protein kinase family molecule, referred to herein as “20750,” is differentially expressed in tumor cells, e.g., colon, lung, and breast tumor cells and in colon cells which have metastasized to the liver, as compared to normal cells. 20750 expression is also upregulated in colon tumors in Smad3^{-/-} mouse models and in the G2/M phase of HCT-116 human colon adenocarcinoma cell lines, indicating a role for 20750 in carcinogenesis and cellular proliferation. The Smad3^{-/-} mouse is a useful and unique model for human colorectal carcinogenesis. Smad3^{-/-} mice develop colon carcinomas that histopathologically resemble human disease. Samples from several stages of disease progression can be isolated, including normal epithelium, hyperplastic epithelium, adenomatous polyps, and various degrees of primary carcinoma and lymph node metastases.

The 20750 molecule is a member of the protein kinase family and is homologous to MAP/microtubule affinity-regulating kinase-like 1 (MARKL1) (GenBank Accession No. AB049127). MARKL1 was shown to be regulated by β-catenin in overexpression experiments using the human hepatoma cell line HepG2 (Kato, et al. (2001) Neoplasia 3(1):4-9). Expression levels of MARKL1 were later shown to be markedly elevated in 90% of hepatocellular carcinomas. β-catenin functions in the Wnt signaling pathway, which is described in Miller, et al. (1999) Oncogene 18(55):7860-72 and Peifer (1997) Science 275:1752-1753. Signaling by the Wnt family of secreted growth factors represents one of the major developmentally important signaling pathways controlling cell fate determination, cellular proliferation, cellular migration, and cell polarity. When there is a decrease in β-catenin degradation and an increase in β-catenin concentration in a cell, through, e.g., phosphorylation, Wnt-signaling is increased, leading to increased cellular proliferation and migration, modified cell polarity, and modified cell-fate determination. Inappropriate activation of the Wnt pathway is implicated in a variety of human cancers, most notably colon cancer. Without intending to be limited by mechanism, it is believed that the 20750 molecules, by modulating β-catenin degradation, modulate the Wnt signaling pathway and therefore modulate cellular growth, cellular proliferation, and tumorigenesis.

For example, modulation, e.g., inhibition, of 20750 may modulate, e.g., decrease, the stability of β-catenin by decreasing the phosphorylation of β-catenin. Decreased β-catenin concentration in the cell leads to a decrease in Wnt signaling, thereby leading to decreased cellular growth and proliferation. Accordingly, the 20750 molecules of the present invention provide novel diagnostic targets and therapeutic agents for the treatment, diagnosis, and prognosis of cellular proliferation disorders, e.g., cancer. In a preferred embodiment, the 20750 molecules of the present invention provide novel diagnostic targets and therapeutic agents for the treatment, diagnosis, and prognosis of colon cancer, lung cancer, and breast cancer.

As used herein, a “cellular proliferation disorder” includes a disease or disorder that affects a cellular growth, differentiation, or proliferation process. As used herein, a “cellular growth, differentiation or proliferation process” is a process by which a cell increases in number, size or content, by which a cell develops a specialized set of characteristics which differ from that of other cells, or by which a cell moves closer to or further from a particular location or stimulus. A cellular growth, differentiation, or proliferation process includes amino acid transport and degradation and other metabolic processes of a cell. A cellular proliferation disorder may be characterized by aberrantly regulated cellular growth, proliferation, differentiation, or migration. Cellular proliferation disorders include tumorigenic disease or disorders. As used herein, a “tumorigenic disease or disorder” includes a disease or disorder characterized by aberrantly regulated cellular growth, proliferation, differentiation, adhesion, or migration, which may result in the production of or tendency to produce tumors. As used herein, a “tumor” includes a normal benign or malignant mass of tissue. Examples of cellular growth or proliferation disorders include, but are not limited to, cancer, e.g., carcinoma, sarcoma, or leukemia, examples of which include, but are not limited to, colon, ovarian, lung, breast, endometrial, uterine, hepatic, gastrointestinal, prostate, and brain cancer; tumorigenesis and metastasis; skeletal dysplasia; and hematopoietic and/or myeloproliferative disorders.

“Differential expression”, as used herein, includes both quantitative as well as qualitative differences in the temporal and/or tissue expression pattern of a gene. Thus, a differentially expressed gene may have its expression activated or inactivated in normal versus cellular growth or proliferation disease states. The degree to which expression differs in normal versus cellular growth or proliferation disease states or control versus experimental states need only be large enough to be visualized via standard characterization techniques, e.g., quantitative PCR, Northern analysis, Taqman™ analysis, transcriptional profiling, or subtractive hybridization. The expression pattern of a differentially expressed gene may be used as part of a prognostic or diagnostic cellular proliferation disorder evaluation, or may be used in methods for identifying compounds useful for the treatment of cellular proliferation disorder. In addition, a differentially expressed gene involved in cellular proliferation disorders may represent a target gene such that modulation of the expression level of this gene or the activity of the gene product may act to ameliorate a cellular growth or proliferation disorder. Compounds that modulate target gene expression or activity of the target gene product can be used in the treatment of cellular proliferation disorders. Although the 20750 genes described herein may be differentially expressed with respect to cellular proliferation disorders, and/or their products may interact with gene products important to cellular proliferation disorders, the genes may also be involved in mechanisms important to additional tumor cell processes.

As used interchangeably herein, “20750 activity,” “biological activity of 20750” or “functional activity of 20750,” includes an activity exerted by a 20750 protein, polypeptide or nucleic acid molecule on a 20750 responsive cell or tissue, e.g., a tumor cell, or on a 20750 protein substrate, as determined in vivo, or in vitro, according to standard techniques. 20750 activity can be a direct activity, such as an association with a 20750-target molecule. As used herein, a “substrate” or “target molecule” or “binding partner” is a molecule with which a 20750 protein binds or interacts in nature, such that 20750-mediated function, e.g., modulation of β-catenin degradation or Wnt signaling, is achieved. A 20750 target molecule can be a non-20750 molecule or a 20750 protein or polypeptide. Examples of such target molecules include proteins in the same signaling path as the 20750 protein, e.g., proteins which may function upstream (including both stimulators and inhibitors of activity) or downstream of the 20750 protein in a pathway involving regulation of cellular growth, proliferation or differentiation. Alternatively, a 20750 activity is an indirect activity, such as a cellular signaling activity mediated by interaction of the 20750 protein with a 20750 target molecule. The biological activities of 20750 are described herein. For example, the 20750 proteins can have one or more of the following activities: (1) modulation of the phosphorylation state of 20750 target molecules (e.g., a kinase molecule) or the phosphorylation state of one or more proteins involved in cellular growth, metabolism, or differentiation, e.g., tumor cell growth or differentiation, as described in, for example, Lodish H. et al. Molecular Cell Biology, (Scientific American Books Inc., New York, N.Y., 1995) and Stryer L., Biochemistry, (W. H. Freeman, New York), the contents of which are incorporated herein by reference; (2) modulation of the activity of one or more proteins involved in cellular growth or differentiation, e.g., tumor cell growth or differentiation; (3) modulation of expression of one or more genes (e.g., a transcription factor), and (4) modulation of signal transduction. In other preferred embodiments, the 20750 polypeptides of the present invention have one or more of the following activities: (1) modulation of cancer or tumor progression, (2) modulation of cellular proliferation, (4) modulation of cellular differentiation, (5) modulation of cellular migration, (6) modulation of apoptosis, (7) modulation of cell polarity, (8) modulation of β-catenin stability, e.g., degradation or accumulation in a cell, and (9) modulation of the Wnt signaling pathway.

Various aspects of the invention are described in further detail in the following subsections:

I. Screening Assays

The invention provides methods (also referred to herein as “screening assays”) for identifying modulators, i.e., candidate or test compounds or agents (e.g., peptides, peptidomimetics, small molecules, ribozymes, or 20750 antisense molecules) which bind to 20750 proteins, have a stimulatory or inhibitory effect on 20750 expression or 20750 activity, or have a stimulatory or inhibitory effect on the expression or activity of a 20750 target molecule. Compounds identified using the assays described herein may be useful for treating cellular proliferation disorders.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

Assays that may be used to identify compounds that modulate 20750 activity include assays that determine the ability of 20750 to phosphorylate a target molecule. 20750 activity can be determined by, for example, using an in vitro kinase assay. Briefly, a kinase target molecule, e.g., β-catenin, can be incubated with the kinase protein and radioactive ATP, e.g., [γ-³²P] ATP, in a buffer containing MgCl₂ and MnCl₂, e.g., 10 mM MgCl₂ and 5 mM MnCl₂. Following the incubation, the immunoprecipitated kinase target molecule can be separated by SDS-polyacrylamide gel electrophoresis under reducing conditions, transferred to a membrane, e.g., a PVDF membrane, and autoradiographed. The appearance of detectable bands on the autoradiograph indicates that the kinase substrate has been phosphorylated. Phosphoaminoacid analysis of the phosphorylated substrate can also be performed in order to determine which residues on the kinase substrate are phosphorylated. Briefly, the radiophosphorylated protein band can be excised from the SDS gel and subjected to partial acid hydrolysis. The products can then be separated by one-dimensional electrophoresis and analyzed on, for example, a phosphoimager and compared to ninhydrin-stained phosphoaminoacid standards. Modulation of β-catenin stability can be determined by assaying for phosphorylation of β-catenin by, for example, methods described in Moon, et al.(2001) Gynecol Oncol. 81(3)355-9. Modulation of the Wnt signaling pathway can be determined by assaying for β-catenin concentration in the cell as described in, for example, Wong, et al.(2001) Cancer 92(1):136-145. Other assays that may be used to identify compounds that modulate 20750 activity include assays to determine (1) modulation of the activity of one or more proteins involved in cellular growth or differentiation, e.g., tumor cell growth or differentiation; (2) modulation of expression of one or more genes (e.g., a transcription factor), (3) modulation of signal transduction, (4) modulation of cancer or tumor progression, (5) modulation of cellular proliferation, (6) modulation of cellular differentiation, (7) modulation of cellular migration, (8) modulation of apoptosis, (9) and modulation of cell polarity.

Cellular proliferation assays that may be used to identify compounds that modulate 20750 activity include assays such as the acid phosphatase assay for cell number as described in Connolly et al. (1986) Anal. Biochem. 152, 136-140 and the MTT assay as described in Loveland, B. E. et al., (1992) Biochem. Int., 27:501-510, which utilizes colorimetric assays to quantitate viable cells, e.g., the cellular reduction of the tetrazolium salt, MTT, to formazan by mitochondrial succinate dehydrogenase. Other assays for cellular proliferation include clonogenic assays, assays for ³H-thymidine uptake, assays measuring the incorporation of radioactively labeled nucleotides into DNA, or other assays which are known in the art for measuring cellular proliferation. Moreover, inhibition of cellular growth in vivo, e.g., in a patient with cancer, can be detected by any standard method for detecting tumors such as by x-ray or imaging analysis of a tumor size, or by observing a reduction in mutant p53 protein production or in the production of any known cell-specific or tumor marker within a biopsy or tissue sample. Determining the ability of a test compound to modulate 20750 activity can be accomplished by monitoring, for example, cell progression through the cell cycle. For example, the cell can be a tumor cell, e.g., a colon tumor cell, a lung tumor cell, or a breast tumor cell.

In one aspect, an assay is a cell-based assay in which a cell which expresses a 20750 protein or biologically active portion thereof is contacted with a test compound and the ability of the test compound to modulate 20750 activity is determined. In a preferred embodiment, the biologically active portion of the 20750 protein includes a domain or motif that can modulate amino acid transport or degradation, cellular metabolism, or cellular growth or proliferation. Determining the ability of the test compound to modulate 20750 activity can be accomplished by monitoring, for example, the production of one or more specific metabolites in a cell which expresses 20750 (see, e.g., Saada et al. (2000) Biochem Biophys. Res. Commun. 269: 382-386) or by monitoring cell metabolism, cellular growth, cellular proliferation, or cellular differentiation. The cell, for example, can be of mammalian origin, e.g., a tumor cell such as a lung, breast, or colon tumor cell.

The ability of the test compound to modulate 20750 binding to a substrate or to bind to 20750 can also be determined. Determining the ability of the test compound to modulate 20750 binding to a substrate can be accomplished, for example, by coupling the 20750 substrate with a radioisotope or enzymatic label such that binding of the 20750 substrate to 20750 can be determined by detecting the labeled 20750 substrate in a complex. Alternatively, 20750 could be coupled with a radioisotope or enzymatic label to monitor the ability of a test compound to modulate 20750 binding to a 20750 substrate in a complex. Determining the ability of the test compound to bind 20750 can be accomplished, for example, by coupling the compound with a radioisotope or enzymatic label such that binding of the compound to 20750 can be determined by detecting the labeled 20750 compound in a complex. For example, 20750 substrates can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to interact with 20750 without the labeling of any of the interactants. For example, a microphysiometer can be used to detect the interaction of a compound with 20750 without the labeling of either the compound or the 20750 molecule (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 20750.

The ability of a 20750 modulator to modulate, e.g., inhibit or increase, 20750 activity can also be determined through screening assays which identify modulators which either increase or decrease amino acid transport or degradation, cellular metabolism, cellular growth, or cellular proliferation. In one embodiment, the invention provides for a screening assay involving contacting cells which express a 20750 protein or polypeptide with a test compound, and examining the cells for cellular growth or proliferation. For example, cells expressing a 20750 protein or polypeptide can be contacted with a test compound and subsequently quantitated to measure cellular growth and/or proliferation as described in, for example, Loveland, B. E. et al, (1992) Biochem. Int., 27:501-510, or by measuring the incorporation of radioactively labeled nucleotides into DNA, or by measuring the number of cells present compared to a control cell. The number of cells can be measured, for example, by dry/wet weight measurement, by counting the cells via optical density, using a counting chamber, or by using other assays for cellular proliferation as described herein or known in the art.

Because 20750 expression is increased in tumors, including metastatic tumors, and is regulated during the cell cycle, compounds which modulate cellular proliferation, growth, and/or differentiation can be identified by the ability to modulate 20750 expression. To determine whether a test compound modulates 20750 expression, a cell which expresses 20750 (e.g., a lung tumor cell, an breast tumor cell, a colon tumor cell, or a corresponding normal cell) is contacted with a test compound, and the ability of the test compound to modulate 20750 expression can be determined by measuring 20750 mRNA by, e.g., Northern Blotting, quantitative PCR (e.g., Taqman), or in vitro transcriptional assays. To perform an in vitro transcriptional assay, the full length promoter and enhancer of 20750 can be linked to a reporter gene such as chloramphenicol acetyltransferase (CAT) or luciferase and introduced into host cells. The same host cells can then be transfected with or contacted with the test compound. The effect of the test compound can be measured by reporter gene activity and comparing it to reporter gene activity in cells which do not contain the test compound. An increase or decrease in reporter gene activity indicates a modulation of 20750 expression and is, therefore, an indicator of the ability of the test compound to modulate cellular proliferation, growth, and/or differentiation in, e.g., tumor cells.

The above described assay for testing the ability of a test compound to modulate 20750 expression can also be used to test the ability of the 20750 molecule to modulate cellular proliferation. If a test compound can modulate 20750 expression it can most likely modulate the cellular growth or proliferation, e.g., tumor cellular growth or proliferation.

In vitro cell-based models for cancer may also be used to identify compounds that modulate 20750 activity and/or to confirm the ability of the test compound to modulate the activity of a 20750 molecule. For example, cell lines may be transiently or stably transfected with tumor suppressors and oncogenes, e.g., including, but not limited, to wild type or mutated p53, Smad4, p16, p14, c-myc, and k-ras, which are genes known to be associated with cancer progression or inhibition, e.g., colon, lung, breast, or ovarian cancer progression or inhibition. These cell lines can then be used to evaluate expression or activity of 20750 in the presence or absence of a test compound using the methods described herein. For example, the following human mammary epithelial cell lines are available for use in in vitro models and/or in xenograft models in mice: HMEC, MCF-7, T-47D, ZR-75, MDA-MB-231, MDA-MB-MC-2, MDA-MB-435, BT-549, SkBr3, MDA-MB-468, MCF10A, MCF10AT.cl1, MCF10AT.cl3, MCF10AT1, MCF10AT3B, MCF10CA1.cl, Hs578T, and HCC1937. The following colon cell lines are available for use in in vitro models and/or in xenograft models in mice: HCT-116, SW480, CC-ML3, KM12C, KM12SM, HT29, DLD-1, HCC-2998, COLO-205, HCT-15, SW-620, and KM20L2. The following lung cell lines are available for use in in vitro models and/or in xenograft models in mice: NCI-H345, NCI-H69, and NCI-H125. The following ovarian cell lines are available for use in in vitro models and/or in xenograft models in mice: SKOV3, SKOV3, OVCAR-3, and OVCAR-4

In vitro cell-based models for breast cancer include, for example, the MCF10A cell line transformed with k-ras, a cell-based system of mammary epithelial malignancy; treating human breast epithelial cells (MCF10A) with growth factors, including EGF and IGF1 growth factors; and reintroduction of BRCA1 expression into HCC1937 cells.

In vitro cell-based models for ovarian cancer include, for example, treatment of the ovarian cancer cell lines, SKOV3 and SKOV3/Variant (which are a variant of the parental SKOV3 ovarian cancer cell line that are cisplatin resistant), with either Epidermal Growth Factor (EGF) or the growth factor Heregulin (Hrg) for 15, 30 and 60 minutes in the absence of serum; and stable expression of p53 in a previously null cell line (SKOV-3 and SKOV3-Var).

In vitro cell-based models for lung cancer include, for example, tumor suppressor models such as reintroduction of p53 into NCI-H125 cells, a lung tumor cell line that is null for p53; expression of p16 and p14, distinct tumor suppressors derived from the same genetic locus, both of which are commonly silenced in lung tumors, in the lung tumor cell lines NCI-H460 and A549, which normally lack expression of these genes; and expression of the pRb gene, which is commonly deleted in small cell lung cancer in small cell tumor lines. Other cell-based models include a stably transformed bronchial epithelial cell line with activated k-ras gene. In addition, growth factor models may also be used. For example, NCI-H69 and NCI-H345 small cell lung carcinoma (SCLC) cells may be treated with a substance P analogue (SPA) that acts as a broad spectrum neuropeptide receptor inhibitor. Genes that were downregulated after SPA treatment were flagged for further study to determine if their expression is critical for tumor cell proliferation. SCLC cells that express both the c-kit tyrosine kinase receptor and its ligand, SCF, may be treated with the kinase inhibitor STI-571. It has been demonstrated that selective growth inhibition upon 571 treatment of cell lines expressing both the receptor and ligand, suggesting that they function in an autocrine feedback loop to stimulate tumor cell proliferation. In vitro cell-based models for cancer may also be used to identify compounds that modulate 20750 activity and/or to confirm the ability of the test compound to modulate the activity of a 20750 molecule. For example, cell lines may be transiently or stably transfected with tumor suppressors and oncogenes, e.g., including, but not limited, to wild type or mutated p53, Smad4, p16, p14, c-myc, and k-ras, which are genes known to be associated with cancer progression or inhibition, e.g., colon, lung, breast, or ovanan cancer progression or inhibition. These cell lines can then be used to evaluate expression or activity of 20750 in the presence or absence of a test compound using the methods described herein. For example, the following human mammary epithelial cell lines are available for use in in vitro models and/or in xenograft models in mice: HMEC, MCF-7, T-47D, ZR-75, MDA-MB-231, MDA-MB-MC-2, MDA-MB-435, BT-549, SkBr3, MDA-MB-468, MCF10A, MCF10AT.cl11, MCF10AT.cl3, MCF10AT1, MCF10AT3B, MCF10CA1.cl, Hs578T, and HCC1937. The following colon cell lines are available for use in in vitro models and/or in xenograft models in mice: HCT-116, SW480, CC-ML3, KM12C, KM12SM, HT29, DLD-1, HCC-2998, COLO-205, HCT-15, SW-620, and KM20L2. The following lung cell lines are available for use in in vitro models and/or in xenograft models in mice: NCI-H345, NCI-H69, and NCI-H125. The following ovarian cell lines are available for use in in vitro models and/or in xenograft models in mice: SKOV3, SKOV3, OVCAR-3, and OVCAR-4

In vitro cell-based models for breast cancer include, for example, the MCF10A cell line transformed with k-ras, a cell-based system of mammary epithelial malignancy; treating human breast epithelial cells (MCF10A) with growth factors, including EGF and IGF1 growth factors; and reintroduction of BRCA1 expression into HCC1937 cells.

In vitro cell-based models for ovarian cancer include, for example, treatment of the ovarian cancer cell lines, SKOV3 and SKOV3/Variant (which are a variant of the parental SKOV3 ovarian cancer cell line that are cisplatin resistant), with either Epidermal Growth Factor (EGF) or the growth factor Heregulin (Hrg) for 15, 30 and 60 minutes in the absence of serum; and stable expression of p53 in a previously null cell line (SKOV-3 and SKOV3-Var).

In vitro cell-based models for lung cancer include, for example, tumor suppressor models such as reintroduction of p53 into NCI-H125 cells, a lung tumor cell line that is null for p53; expression of p16 and p14, distinct tumor suppressors derived from the same genetic locus, both of which are commonly silenced in lung tumors, in the lung tumor cell lines NCI-H460 and A549, which normally lack expression of these genes; and expression of the pRb gene, which is commonly deleted in small cell lung cancer in small cell tumor lines. Other cell-based models include a stably transformed bronchial epithelial cell line with activated k-ras gene. In addition, growth factor models may also be used. For example, NCI-H69 and NCI-H345 small cell lung carcinoma (SCLC) cells may be treated with a substance P analogue (SPA) that acts as a broad spectrum neuropeptide receptor inhibitor. Genes that were downregulated after SPA treatment were flagged for further study to determine if their expression is critical for tumor cell proliferation. SCLC cells that express both the c-kit tyrosine kinase receptor and its ligand, SCF, may be treated with the kinase inhibitor STI-571. It has been demonstrated that selective growth inhibition upon 571 treatment of cell lines expressing both the receptor and ligand, suggesting that they function in an autocrine feedback loop to stimulate tumor cell proliferation.

In vitro cell-based models for colon cancer include, for example, SW480 cells stably or transiently transfected with Smad4. Smad4 is a candidate tumor suppressor gene mutated in a subset of colon carcinomas. Smad4 functions in the signal transduction of TGF-β molecules. It is well known that the TGF-β superfamily is involved in growth inhibition. Smad4 mutation/loss in colon cell lines provides the hypothesis that Smad4 may be a modulator of cell adhesion and invasion. Another cell line useful in the methods of the invention are NCM425 cells stably or transiently transfected with β-catenin. Mutations of the APC gene are responsible for tumor formation in sporadic and familial forms of colorectal cancer. APC binds β-catenin and regulates the cytoplasmic levels of β-catenin. When APC is mutated, β-catenin accumulates in the cytoplasm and translocates into the nucleus. Once in the nucleus it interacts with LEF/TCF molecules and regulates gene expression. Genes regulated by the β-catenin/LEF complex, like c-myc and cyclin D1, are involved in tumorigenesis. Also useful in the methods of the invention are cells stably or transiently transfected with p53. p53 is a well-known tumor suppressor which is mutated in >50% of colorectal cancer tumors. Still other cell lines useful in the methods of the invention include transient or stable transfections of WISP-1 into NCM425 colon cancer cells, transient or stable transfections of DCC, Cox2, and/or APC into various cells.

Cell lines such as HCT-116 and DLD-1 may also be transformed with k-ras and used in the method of the invention. Point mutations that activate the k-ras oncogene are found in 50% of human colon cancers. Activated k-ras may regulate cell proliferation in colorectal tumors. Disrupting the activated k-ras allele in HCT-116 and DLD-1 cells morphologically alters differentiation, causes loss of anchorage independent growth, slows proliferation in vitro and in vivo and reduces expression of c-myc. Expression of 20750 was found to be downregulated in k-ras disrupted HCT-116 cells.

Abnormalities in cell cycle regulation and its checkpoints lead to the development of malignant cells. The loss of a cell's ability to respond to signals that regulate cell proliferation and cell cycle arrest is a common mechanism of cancer. Accordingly, for the study of specific time point within the cell cycle, cell lines such as the colon cancer cell lines HCT116, DLD-1, and NCM425, for example, may be synchronized with agents such as mimosine (G1 block), mimosine (G1/S block) and nocodazole (G2/M block). Cell synchronization in relation to p53 status may also be studied in cells of varying p⁵³ status (SKOV-3 (null), OVCAR-3 or OVCAR-4 (mutant), and HEY (wildtype)).

In yet another embodiment, an assay of the present invention is a cell-free assay in which a 20750 protein or biologically active portion thereof is contacted with a test compound and the ability of the test compound to bind to or to modulate (e.g., stimulate or inhibit) the activity of the 20750 protein or biologically active portion thereof is determined. Preferred biologically active portions of the 20750 proteins to be used in assays of the present invention include fragments which participate in interactions with non-20750 molecules, e.g., fragments with high surface probability scores. Binding of the test compound to the 20750 protein can be determined either directly or indirectly as described above. Determining the ability of the 20750 protein to bind to a test compound can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In more than one embodiment of the above assay methods of the present invention, it may be desirable to immobilize either 20750 or a 20750 target molecule to facilitate separation of complexed from uncomplexed forms of one or both of the proteins, as well as to accommodate automation of the assay. Binding of a test compound to a 20750 protein, or interaction of a 20750 protein with a 20750 target molecule in the presence and absence of a test compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/20750 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 20750 protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of 20750 binding or activity determined using standard techniques.

Other techniques for immobilizing proteins on matrices can also be used in the screening assays of the invention. For example, either a 20750 protein or a 20750 target molecule can be immobilized utilizing conjugation of biotin and streptavidin. Biotinylated 20750 protein or target molecules can be prepared from biotin-NHS (N-hydroxy-succinimide) using techniques known in the art (e.g., biotinylation kit, Pierce Chemicals, Rockford, Ill., and immobilized in the wells of streptavidin-coated 96 well plates (Pierce Chemical). Alternatively, antibodies which are reactive with 20750 protein or target molecules but which do not interfere with binding of the 20750 protein to its target molecule can be derivatized to the wells of the plate, and unbound target or 20750 protein is trapped in the wells by antibody conjugation. Methods for detecting such complexes, in addition to those described above for the GST-immobilized complexes, include immunodetection of complexes using antibodies reactive with the 20750 protein or target molecule, as well as enzyme-linked assays which rely on detecting an enzymatic activity associated with the 20750 protein or target molecule.

In yet another aspect of the invention, the 20750 protein or fragments thereof can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 20750 (“20750-binding proteins” or “20750-bp) and are involved in 20750 activity. Such 20750-binding proteins are also likely to be involved in the propagation of signals by the 20750 proteins or 20750 targets as, for example, downstream elements of a 20750-mediated signaling pathway. Alternatively, such 20750-binding proteins are likely to be 20750 inhibitors.

The two-hybrid system is based on the modular nature of most transcription factors, which consist of separable DNA-binding and activation domains. Briefly, the assay utilizes two different DNA constructs. In one construct, the gene that codes for a 20750 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 20750-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 20750 protein.

In another aspect, the invention pertains to a combination of two or more of the assays described herein. For example, a modulating agent can be identified using a cell-based or a cell-free assay, and the ability of the agent to modulate the activity of a 20750 protein can be confirmed in vivo, e.g., in an animal such as an animal model for a cellular proliferation disorder, e.g., cancer. Examples of animal models of cancer include transplantable models (e.g., xenografts). Xenografts for colon cancer can be performed with the following cell lines: HCT-116, HT-29, SW-480, SW-620, Colon 26, DLD1, Caco2, colo205, T84, and KM12. Xenografts for lung cancer can be performed with the following cell lines: NCI-H125, NCI-H460, A549, NCI-H69, and NCI-H345. Xenografts for ovarian cancer can be performed with the SKOV3 and HEY cell lines. Xenografts for breast cancer can be performed with, for example, MCF10AT cells, which can be grown as subcutaneous or orthotopic (cleared mammary fat pad) xenografts in mice. MCF10AT xenografts produce tumors that progress in a manner analogous to human breast cancer. Estrogen stimulation has also been shown to accelerate tumor progression in this model. MCF10AT xenografted tumors representing stages hyperplasia, carcinoma in situ, and invasive carcinoma will be isolated expression profiling. A metastatic subclone of the human breast cancer cell line MDA-MB-231 that metastasizes to brain, lung and bone can also be grown in vitro and in vivo at various sites (i.e. subcutaneously, orthotopically, in bone following direct bone injection, in bone following intracardiac injection). MCF-7 and T-47D are other mammary adenocarcinoma cell lines that can be grown as xenografts. All of these cells can be transplanted into immunocompromised mice such as SCID or nude mice, for example.

Orthotopic metastasis mouse models may also be utilized. For example, the HCT-116 human colon carcinoma cell line can be grown as a subcutaneous or orthotopic xenograft (intracaecal injection) in athymic nude mice. Rare liver and lung metastases can be isolated, expanded in vitro, and re-implanted in vivo. A limited number of iterations of this process can be employed to isolate highly metastatic variants of the parental cell line. Standard and subtracted cDNA libraries and probes can be generated from the parental and variant cell lines to identify genes associated with the acquisition of a metastatic phenotype. This model can be established using several alternative human colon carcinoma cell lines, including SW480 and KM12C.

Also useful in the methods of the invention are mis-match repair models (MMRs). Hereditary nonpolyposis colon cancer (HNPCC), which is caused by germline mutations in MSH2 & MLH1, genes involved in DNA mismatch repair, accounts for 5-15% of colon cancer cases. Mouse models have been generated carrying null mutations in the MLH1, MSH2 and MSH3 genes.

Other animal models for cancer include transgenic models (e.g., B66-Min/+mouse); chemical induction models, e.g., carcinogen (e.g., azoxymethane, 2-dimethylhydrazine, or N-nitrosodimethylamine) treated rats or mice; models of liver metastasis from colon cancer such as that described by Rashidi et al. (2000) Anticancer Res 20(2A):715; and cancer cell implantation or inoculation models as described in, for example, Fingert et al. (1987) Cancer Res 46(14):3824-9 and Teraoka et al. (1995) Jpn J Cancer Res 86(5):419-23. Furthermore, experimental model systems are available for the study of, for example, ovarian cancer (Hamilton, T C et al. Semin Oncol (1984) 11:285-298; Rahman, N A et al. Mol Cell Endocrinol (1998) 145:167-174; Beamer, W G et al. Toxicol Pathol (1998) 26:704-710), gastric cancer (Thompson, J et al. Int J Cancer (2000) 86:863-869; Fodde, R et al. Cytogenet Cell Genet (1999) 86:105-111), breast cancer (Li, M et al. Oncogene (2000) 19:1010-1019; Green, J E et al. Oncogene (2000) 19:1020-1027), melanoma (Satyamoorthy, K et al. Cancer Metast Rev (1999) 18:401-405), and prostate cancer (Shirai, T et al. Mutat Res (2000) 462:219-226; Bostwick, D G et al. Prostate (2000) 43:286-294). Mouse models for colon cancer include the APC^{nun} mouse, a highly characterized genetic model of human colorectal carcinogeneis; the APC^{1638N} mouse, which was generated by introducing a PGK-neomycin gene at codon 1638 of the APC gene and develops aberrant crypt foli after 6-8 weeks which ultimately progress to carcinomas by 4 months of age; and the Smad3^{-/-} mouse which develops colon carcinomas that histopathologically resemble human disease.

Other animal based models for studying tumorigenesis in vivo are well known in the art (reviewed in Animal Models of Cancer Predisposition Syndromes, Hiai, H. and Hino, O. (eds.) 1999, Progress in Experimental Tumor Research, Vol. 35; Clarke A R Carcinogenesis (2000) 21:435-41) and include, for example, carcinogen-induced tumors (Rithidech, K et al. Mutat Res (1999) 428:33-39; Miller, M L et al. Environ Mol Mutagen (2000) 35:319-327), as well as animals bearing mutations in growth regulatory genes, for example, oncogenes (e.g., ras) (Arbeit, J M et al. Am J Pathol (1993) 142:1187-1197; Sinn, E et al. Cell (1987) 49:465-475; Thorgeirsson, S S et al. Toxicol Lett (2000) 112-113:553-555) and tumor suppressor genes (e.g., p53) (Vooijs, M et al. Oncogene (1999) 18:5293-5303; Clark A R Cancer Metast Rev (1995) 14:125-148; Kumar, T R et al. J Intern Med (1995) 238:233-238; Donehower, L A et al. (1992) Nature 356215-221).

Furthermore, this invention pertains to uses of novel compounds identified by the above-described screening assays for treatments as described herein. In one embodiment, the invention features a method of treating a subject having a cellular growth or proliferation disorder that involves administering to the subject an 20750 modulator such that treatment occurs. In another embodiment, the invention features a method of treating a subject having cancer, e.g., colon cancer, lung cancer, or ovarian cancer, that involves treating a subject with an 20750 modulator, such that treatment occurs. Preferred 20750 modulators include, but are not limited to, 20750 proteins or biologically active fragments, 20750 nucleic acid molecules, 20750 antibodies, ribozymes, and 20750 antisense oligonucleotides designed based on the 20750 nucleotide sequences disclosed herein, as well as peptides, organic and non-organic small molecules identified as being capable of modulating 20750 expression and/or activity, for example, according to at least one of the screening assays described herein.

Moreover, a 20750 modulator identified as described herein (e.g., an antisense 20750 nucleic acid molecule, a 20750-specific antibody, or a small molecule) can be used in an animal model to determine the efficacy, toxicity, or side effects of treatment with such a modulator. Alternatively, a 20750 modulator identified as described herein can be used in an animal model to determine the mechanism of action of such a modulator.

Any of the compounds, including but not limited to compounds such as those identified in the foregoing assay systems, may be tested for the ability to ameliorate cellular growth or proliferation disorder symptoms. Cell-based and animal model-based assays for the identification of compounds exhibiting such an ability to ameliorate cellular growth or proliferation disorder systems are described herein.

In one aspect, cell-based systems, as described herein, may be used to identify compounds which may act to ameliorate cellular growth or proliferation disorder symptoms, for example, reduction in tumor burden, tumor size, tumor cellular growth, differentiation, and/or proliferation, and invasive and/or metastatic potential before and after treatment. For example, such cell systems may be exposed to a compound, suspected of exhibiting an ability to ameliorate cellular growth or proliferation disorder symptoms, at a sufficient concentration and for a time sufficient to elicit such an amelioration of cellular growth or proliferation disorder symptoms in the exposed cells. After exposure, the cells are examined to determine whether one or more of the cellular growth or proliferation disorder cellular phenotypes has been altered to resemble a more normal or more wild type, non-cellular growth or proliferation disorder phenotype. Cellular phenotypes that are associated with cellular growth and/or proliferation disorders include aberrant proliferation, growth, and migration, anchorage independent growth, and loss of contact inhibition.

In addition, animal-based cellular growth or proliferation disorder systems, such as those described herein, may be used to identify compounds capable of ameliorating cellular growth or proliferation disorder symptoms. Such animal models may be used as test substrates for the identification of drugs, pharmaceuticals, therapies, and interventions which may be effective in treating cellular growth or proliferation disorders. For example, animal models may be exposed to a compound, suspected of exhibiting an ability to ameliorate cellular growth or proliferation disorder symptoms, at a sufficient concentration and for a time sufficient to elicit such an amelioration of cellular growth or proliferation disorder symptoms in the exposed animals. The response of the animals to the exposure may be monitored by assessing the reversal of cellular growth or proliferation disorders, or symptoms associated therewith, for example, reduction in tumor burden, tumor size, and invasive and/or metastatic potential before and after treatment.

With regard to intervention, any treatments which reverse any aspect of cellular growth or proliferation disorder symptoms should be considered as candidates for human cellular growth or proliferation disorder therapeutic intervention. Dosages of test compounds may be determined by deriving dose-response curves.

Additionally, gene expression patterns may be utilized to assess the ability of a compound to ameliorate cellular growth and/or proliferation disorder symptoms. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Such conditions may include, but are not limited to, cellular growth, proliferation, differentiation, transformation, tumorigenesis, metastasis, and carcinogen exposure. Gene expression profiles may be generated, for example, by utilizing a differential display procedure, Northern analysis and/or RT-PCR. In one embodiment, 20750 gene sequences may be used as probes and/or PCR primers for the generation and corroboration of such gene expression profiles.

Gene expression profiles may be characterized for known states within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of a cellular growth or proliferation disorder model system to more closely resemble the control system. Administration of a compound may, alternatively, cause the gene expression profile of a control system to begin to mimic a cellular growth and/or proliferation disorder state. Such a compound may, for example, be used in further characterizing the compound of interest, or may be used in the generation of additional animal models.

II. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 20750 protein and/or nucleic acid expression as well as 20750 activity, in the context of a biological sample (e.g., blood, serum, cells, or tissue, e.g., tumor or carcinoma tissue) to thereby determine whether an individual is afflicted with a cellular proliferation disorder. The invention also provides for prognostic (or predictive) assays for determining whether an individual is at risk of developing a cellular proliferation disorder. For example, mutations in a 20750 gene can be assayed for in a biological sample. Such assays can be used for prognostic or predictive purpose to thereby phophylactically treat an individual prior to the onset of a cellular proliferation disorder.

Another aspect of the invention pertains to monitoring the influence of 20750 modulators (e.g., anti-20750 antibodies or 20750 ribozymes) on the expression or activity of 20750 in clinical trials.

These and other agents are described in further detail in the following sections.

A. Diagnostic Assays for Cellular Proliferation Disorders

To determine whether a subject is afflicted with a cellular proliferation disorder, a biological sample may be obtained from a subject and the biological sample may be contacted with a compound or an agent capable of detecting a 20750 protein or nucleic acid (e.g., mRNA or genomic DNA) that encodes a 20750 protein, in the biological sample. A preferred agent for detecting 20750 mRNA or genomic DNA is a labeled nucleic acid probe capable of hybridizing to 20750 mRNA or genomic DNA. The nucleic acid probe can be, for example, the 20750 nucleic acid set forth in SEQ ID NO:1, or a portion thereof, such as an oligonucleotide of at least 15, 20, 25, 30, 25, 40, 45, 50, 100, 250 or 500 nucleotides in length and sufficient to specifically hybridize under stringent conditions to 20750 mRNA or genomic DNA. Other suitable probes for use in the diagnostic assays of the invention are described herein.

A preferred agent for detecting 20750 protein in a sample is an antibody capable of binding to 20750 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect 20750 mRNA, protein, or genomic DNA in a biological sample in vitro as well as in vivo. For example, in vitro techniques for detection of 20750 mRNA include Northern hybridizations and in situ hybridizations. In vitro techniques for detection of 20750 protein include enzyme linked immunosorbent assays (ELISAs), Western blots, immunoprecipitations and immunofluorescence. In vitro techniques for detection of 20750 genomic DNA include Southern hybridizations. Furthermore, in vivo techniques for detection of 20750 protein include introducing into a subject a labeled anti-20750 antibody. For example, the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting 20750 protein, mRNA, or genomic DNA, such that the presence of 20750 protein, mRNA or genomic DNA is detected in the biological sample, and comparing the presence of 20750 protein, mRNA or genomic DNA in the control sample with the presence of 20750 protein, mRNA or genomic DNA in the test sample.

B. Prognostic Assays for Cellular Proliferation Disorders

The present invention further pertains to methods for identifying subjects having or at risk of developing a cellular proliferation disorder associated with aberrant 20750 expression or activity.

As used herein, the term “aberrant” includes a 20750 expression or activity which deviates from the wild type 20750 expression or activity. Aberrant expression or activity includes increased or decreased expression or activity, as well as expression or activity which does not follow the wild type developmental pattern of expression or the subcellular pattern of expression. For example, aberrant 20750 expression or activity is intended to include the cases in which a mutation in the 20750 gene causes the 20750 gene to be under-expressed or over-expressed and situations in which such mutations result in a non-functional 20750 protein or a protein which does not function in a wild-type fashion, e.g., a protein which does not interact with a 20750 substrate, or one which interacts with a non-20750 substrate.

The assays described herein, such as the preceding diagnostic assays or the following assays, can be used to identify a subject having or at risk of developing a cellular proliferation disorder, e.g., cancer, such as for example, colon, lung, and ovarian cancer. A biological sample may be obtained from a subject and tested for the presence or absence of a genetic alteration. For example, such genetic alterations can be detected by ascertaining the existence of at least one of 1) a deletion of one or more nucleotides from a 20750 gene, 2) an addition of one or more nucleotides to a 20750 gene, 3) a substitution of one or more nucleotides of a 20750 gene, 4) a chromosomal rearrangement of a 20750 gene, 5) an alteration in the level of a messenger RNA transcript of a 20750 gene, 6) aberrant modification of a 20750 gene, such as of the methylation pattern of the genomic DNA, 7) the presence of a non-wild type splicing pattern of a messenger RNA transcript of a 20750 gene, 8) a non-wild type level of a 20750-protein, 9) allelic loss of a 20750 gene, and 10) inappropriate post-translational modification of a 20750-protein.

As described herein, there are a large number of assays known in the art which can be used for detecting genetic alterations in a 20750 gene. For example, a genetic alteration in a 20750 gene may be detected using a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a 20750 gene (see Abravaya et al. (1995) Nucleic Acids Res. 23:675-682). This method includes collecting a biological sample from a subject, isolating nucleic acid (e.g., genomic DNA, mRNA or both) from the sample, contacting the nucleic acid sample with one or more primers which specifically hybridize to a 20750 gene under conditions such that hybridization and amplification of the 20750 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 20750 gene from a biological sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 20750 can be identified by hybridizing biological sample derived and control nucleic acids, e.g., DNA or RNA, to high density arrays containing hundreds or thousands of oligonucleotide probes (Cronin, M. T. et al. (1996) Human Mutation 7:244-255; Kozal, M. J. et al. (1996) Nature Medicine 2:753-759). For example, genetic mutations in 20750 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential, overlapping probes. This step allows for the identification of point mutations. This step is followed by a second hybridization array that allows for the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 20750 gene in a biological sample and detect mutations by comparing the sequence of the 20750 in the biological sample with the corresponding wild-type (control) sequence. Examples of sequencing reactions include those based on techniques developed by Maxam and Gilbert (1977) Proc. Natl. Acad. Sci. USA 74:560) or Sanger (1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 20750 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type 20750 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 20750 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 20750 sequence, e.g., a wild-type 20750 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Patent No. 5,459,039.

In other embodiments, alterations in electrophoretic mobility will be used to identify mutations in 20750 genes. For example, single strand conformation polymorphism (SSCP) may be used to detect differences in electrophoretic mobility between mutant and wild type nucleic acids (Orita et al. (1989) Proc Natl. Acad. Sci USA: 86:2766; see also Cotton (1993) Mutat. Res. 285:125-144 and Hayashi (1992) Genet. Anal. Tech. Appl. 9:73-79). Single-stranded DNA fragments of sample and control 20750 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. ( 1 991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Furthermore, the prognostic assays described herein can be used to determine whether a subject can be administered a 20750 modulator (e.g., an agonist, antagonist, peptidomimetic, protein, peptide, nucleic acid, or small molecule) to effectively treat a cellular proliferation disorder.

C. Monitoring of Effects During Clinical Trials

The present invention further provides methods for determining the effectiveness of a 20750 modulator (e.g., a 20750 modulator identified herein) in treating a cellular proliferation disorder in a subject. For example, the effectiveness of a 20750 modulator in increasing 20750 gene expression, protein levels, or in upregulating 20750 activity, can be monitored in clinical trials of subjects exhibiting decreased 20750 gene expression, protein levels, or downregulated 20750 activity. Alternatively, the effectiveness of a 20750 modulator in decreasing 20750 gene expression, protein levels, or in downregulating 20750 activity, can be monitored in clinical trials of subjects exhibiting increased 20750 gene expression, protein levels, or 20750 activity. In such clinical trials, the expression or activity of a 20750 gene, and preferably, other genes that have been implicated in, for example, a cellular proliferation disorder can be used as a “read out” or marker of the phenotype of a particular cell.

For example, and not by way of limitation, genes, including 20750, that are modulated in cells by treatment with an agent which modulates 20750 activity (e.g., identified in a screening assay as described herein) can be identified. Thus, to study the effect of agents which modulate 20750 activity on subjects suffering from a cellular proliferation disorder in, for example, a clinical trial, cells can be isolated and RNA prepared and analyzed for the levels of expression of 20750 and other genes implicated in the cellular proliferation disorder. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of 20750 or other genes. In this way, the gene expression pattern can serve as a marker, indicative of the physiological response of the cells to the agent which modulates 20750 activity. This response state may be determined before, and at various points during treatment of the individual with the agent which modulates 20750 activity.

In a preferred embodiment, the present invention provides a method for monitoring the effectiveness of treatment of a subject with an agent which modulates 20750 activity (e.g., an agonist, antagonist, peptidomimetic, protein, peptide, nucleic acid, or small molecule identified by the screening assays described herein) including the steps of (i) obtaining a pre-administration sample from a subject prior to administration of the agent; (ii) detecting the level of expression of a 20750 protein, mRNA, or genomic DNA in the pre-administration sample; (iii) obtaining one or more post-administration samples from the subject; (iv) detecting the level of expression or activity of the 20750 protein, mRNA, or genomic DNA in the post-administration samples; (v) comparing the level of expression or activity of the 20750 protein, mRNA, or genomic DNA in the pre-administration sample with the 20750 protein, mRNA, or genomic DNA in the post administration sample or samples; and (vi) altering the administration of the agent to the subject accordingly. For example, increased administration of the agent may be desirable to increase the expression or activity of 20750 to higher levels than detected, i.e., to increase the effectiveness of the agent. Alternatively, decreased administration of the agent may be desirable to decrease expression or activity of 20750 to lower levels than detected, i.e. to decrease the effectiveness of the agent. According to such an embodiment, 20750 expression or activity may be used as an indicator of the effectiveness of an agent, even in the absence of an observable phenotypic response.

III. Methods of Treatment of Subjects Suffering from Cellular Proliferation Disorders

The present invention provides for both prophylactic and therapeutic methods of treating a subject, e.g., a human, at risk of (or susceptible to) a cellular proliferation disorder such as cancer, e.g., colon, lung, or ovarian cancer. The term “treatment”, as used herein, is defined as the application or administration of a therapeutic agent to a patient, or application or administration of a therapeutic agent to an isolated tissue or cell line from a patient, who has a disease or disorder, a symptom of a disease or disorder, or a predisposition toward a disease or disorder, with the purpose to cure, heal, alleviate, relieve, alter, remedy, ameliorate, improve or affect the disease or disorder, the symptoms of the disease or disorder, or the predisposition toward a disease or disorder, e.g., the cellular proliferation disorder. A therapeutic agent includes, but is not limited to, small molecules, peptides, antibodies, ribozymes and antisense oligonucleotides.

With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring an subject's prophylactic or therapeutic treatment with either the 20750 molecules of the present invention or 20750 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a cellular proliferation disorder by administering to the subject an agent which modulates 20750 expression or 20750 activity, e.g., modulation of cellular proliferation, e.g., tumor cellular proliferation. Subjects at risk for a cellular proliferation disorder can be identified by, for example, any or a combination of the diagnostic or prognostic assays described herein. Administration of a prophylactic agent can occur prior to the manifestation of symptoms characteristic of aberrant 20750 expression or activity, such that a cellular proliferation disorder is prevented or, alternatively, delayed in its progression. Depending on the type of 20750 aberrancy, for example, a 20750, 20750 agonist or 20750 antagonist agent can be used for treating the subject. The appropriate agent can be determined based on screening assays described herein.

B. Therapeutic Methods

Another aspect of the invention pertains to methods for treating a subject suffering from a cellular proliferation disorder. These methods involve administering to a subject an agent which modulates 20750 expression or activity (e.g., an agent identified by a screening assay described herein), or a combination of such agents. In another embodiment, the method involves administering to a subject a 20750 protein or nucleic acid molecule as therapy to compensate for reduced, aberrant, or unwanted 20750 expression or activity. Modulation, e.g., inhibition of 20750 activity is desirable in situations in which 20750 is abnormally upregulated and/or in which decreased 20750 activity is likely to have a beneficial effect, e.g., inhibition of β-catenin degradation, cellular growth, migration and proliferation, thereby ameliorating a cellular proliferation disorder such as cancer, e.g., colon, lung, or breast cancer, in a subject.

The agents which modulate 20750 activity can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates 20750 activity (e.g., a fragment of a 20750 protein or an anti-20750 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate 20750 activity can also be prepared in the form of suppositories (e.g., with conventional suppository bases such as cocoa butter and other glycerides) or retention enemas for rectal delivery.

In one embodiment, the agents that modulate 20750 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates 20750 activity and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such 20750 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

C. Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent which modulates 20750 activity, as well as tailoring the dosage and/or therapeutic regimen of treatment with an agent which modulates 20750 activity.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a 20750 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a 20750 molecule or 20750 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from a cellular proliferation disorder with an agent which modulates 20750 activity.

IV. Recombinant Expression Vectors and Host Cells Used in the Methods of the Invention

The methods of the invention (e.g., the screening assays described herein) include the use of vectors, preferably expression vectors, containing a nucleic acid encoding a 20750 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 20750 proteins, mutant forms of 20750 proteins, fusion proteins, and the like).

The recombinant expression vectors to be used in the methods of the invention can be designed for expression of 20750 proteins in prokaryotic or eukaryotic cells. For example, 20750 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 20750 activity assays, (e.g., direct assays or competitive assays described in detail below), or to generate antibodies specific for 20750 proteins. In a preferred embodiment, a 20750 fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 20750 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a 20750 nucleic acid molecule of the invention is introduced, e.g., a 20750 nucleic acid molecule within a recombinant expression vector or a 20750 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 20750 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a 20750 protein. Accordingly, the invention further provides methods for producing a 20750 protein using the host cells of the invention. In one embodiment, the method comprises culturing the host cell of the invention (into which a recombinant expression vector encoding a 20750 protein has been introduced) in a suitable medium such that a 20750 protein is produced. In another embodiment, the method further comprises isolating a 20750 protein from the medium or the host cell.

V. Isolated Nucleic Acid Molecules of the Invention

The coding sequence of the isolated human 20750 eDNA and the predicted amino acid sequence of the human 20750 polypeptide are shown in SEQ ID NOs:1 and 2, respectively.

The methods of the invention include the use of isolated nucleic acid molecules that encode 20750 proteins or biologically active portions thereof, as well as nucleic acid fragments sufficient for use as hybridization probes to identify 20750-encoding nucleic acid molecules (e.g., 20750 mRNA) and fragments for use as PCR primers for the amplification or mutation of 20750 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

In one embodiment, the 20750 molecules of the present invention include at least one “transmembrane domain.” As used herein, the term “transmembrane domain” includes an amino acid sequence of about 20-45 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, alanines, valines, phenylalanines, prolines or methionines. Transmembrane domains are described in, for example, Zagotta W. N. et al., (1996) Annual Rev. Neurosci. 19: 235-263, the contents of which are incorporated herein by reference. Amino acid residues 214-231 of the human 20750 polypeptide (SEQ ID NO:2) comprise a transmembrane domain.

To identify the presence of a transmembrane domain in a 20750 protein, and make the determination that a protein of interest has a particular profile, the amino acid sequence of the protein may be subjected to MEMSAT analysis. A MEMSAT analysis of the 20750 protein set forth as SEQ ID NO:2 results in the identification of a transmembrane domain in the amino acid sequence of human 20750 (SEQ ID NO:2) at about residues 130-147 (having a score of 2.9).

In another embodiment, the 20750 molecules of the present invention include at least one “protein kinase domain”. As used herein, the term “protein kinase domain” includes a protein domain having at least about 150-350 amino acid residues and a bit score of at least 150 when compared against a eukaryotic protein kinase domain Hidden Markov Model (HMM), e.g., PFAM Accession Number PF00069. Preferably, a eukaryotic protein kinase domain includes a protein having an amino acid sequence of about 125-325, 150-300, 190-225 or more preferably about 200 amino acid residues, and a bit score of at least 150, 210, 250 or more preferably, 275.7.

A nucleic acid molecule used in the methods of the present invention, e.g., a nucleic acid molecule having the nucleotide sequence of SEQ ID NO:1, or a portion thereof, can be isolated using standard molecular biology techniques and the sequence information provided herein. Using all or portion of the nucleic acid sequence of SEQ ID NO:1 as a hybridization probe, 20750 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1 can be isolated by the polymerase chain reaction (PCR) using synthetic oligonucleotide primers designed based upon the sequence of SEQ ID NO:1.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to 20750 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, a complement of the nucleotide sequence shown in SEQ ID NO:1, or a portion of any of these nucleotide sequences. A nucleic acid molecule which is complementary to the nucleotide sequence shown in SEQ ID NO:1, is one which is sufficiently complementary to the nucleotide sequence shown in SEQ ID NO:1 such that it can hybridize to the nucleotide sequence shown in SEQ ID NO:1 thereby forming a stable duplex.

In still another preferred embodiment, an isolated nucleic acid molecule used in the methods of the present invention comprises a nucleotide sequence which is at least about 55%, 59%, 60%, 65%, 70%, 75%, 80%, 85%, 90%, 95%, 96%, 97%, 98%, 99% or more identical to the entire length of the nucleotide sequence shown in SEQ ID NO:1 or a portion of any of this nucleotide sequence.

Moreover, the nucleic acid molecules used in the methods of the invention can comprise only a portion of the nucleic acid sequence of SEQ ID NO:1, for example, a fragment which can be used as a probe or primer or a fragment encoding a portion of a 20750 protein, e.g., a biologically active portion of a 20750 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1 of an anti-sense sequence of SEQ ID NO:1 or of a naturally occurring allelic variant or mutant of SEQ ID NO:1. In one embodiment, a nucleic acid molecule used in the methods of the present invention comprises a nucleotide sequence which is greater than 100, 100-200, 200-300, 300-400, 400-500, 500-600, 600-700, 700-800, 800-900, 900-1000, 1000-1100, 1100-1200, 1200-1300, 1300-1400, 1400-1500 or more nucleotides in length and hybridizes under stringent hybridization conditions to a nucleic acid molecule of SEQ ID NO:1.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4×sodium chloride/sodium citrate (SSC), at about 65-70° C. (or hybridization in 4× SSC plus 50% formamide at about 42-50° C.) followed by one or more washes in 1× SSC, at about 65-70° C. A preferred, non-limiting example of highly stringent hybridization conditions includes hybridization in 1× SSC, at about 65-70° C. (or hybridization in 1× SSC plus 50% formamide at about 42-50° C.) followed by one or more washes in 0.3× SSC, at about 65-70° C. A preferred, non-limiting example of reduced stringency hybridization conditions includes hybridization in 4× SSC, at about 50-60° C. (or alternatively hybridization in 6× SSC plus 50% formamide at about 40-45° C.) followed by one or more washes in 2× SSC, at about 50-60° C. Ranges intermediate to the above-recited values, e.g., at 65-70° C. or at 42-50° C. are also intended to be encompassed by the present invention. SSPE (1× SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2× SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a 20750 protein, such as by measuring a level of a 20750-encoding nucleic acid in a sample of cells from a subject e.g., detecting 20750 mRNA levels or determining whether a genomic 20750 gene has been mutated or deleted.

The methods of the invention further encompass the use of nucleic acid molecules that differ from the nucleotide sequence shown in SEQ ID NO:1 due to degeneracy of the genetic code and thus encode the same 20750 proteins as those encoded by the nucleotide sequence shown in SEQ ID NO:1. In another embodiment, an isolated nucleic acid molecule included in the methods of the invention has a nucleotide sequence encoding a protein having an amino acid sequence shown in SEQ ID NO:2.

The methods of the invention further include the use of allelic variants of human 20750, e.g., functional and non-functional allelic variants. Functional allelic variants are naturally occurring amino acid sequence variants of the human 20750 protein that maintain a 20750 activity. Functional allelic variants will typically contain only conservative substitution of one or more amino acids of SEQ ID NO:2, or substitution, deletion or insertion of non-critical residues in non critical regions of the protein. Non-functional allelic variants are naturally occurring amino acid sequence variants of the human 20750 protein that do not have a 20750 activity. Non-functional allelic variants will typically contain a non-conservative substitution, deletion, or insertion or premature truncation of the amino acid sequence of SEQ ID NO:2, or a substitution, insertion or deletion in critical residues or critical regions of the protein.

The methods of the present invention may further use non-human orthologues of the human 20750 protein. Orthologues of the human 20750 protein are proteins that are isolated from non-human organisms and possess the same 20750 activity.

The methods of the present invention further include the use of nucleic acid molecules comprising the nucleotide sequence of SEQ ID NO:1 or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of 20750 (e.g., the sequence of SEQ ID NO:2) without altering the biological activity, whereas an “essential” amino acid residue is required for biological activity. For example, amino acid residues that are conserved among the 20750 proteins of the present invention and other members of the protein kinase family are not likely to be amenable to alteration.

Mutations can be introduced into SEQ ID NO:1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 20750 protein is preferably replaced with another amino acid residue from the same side chain family. Alternatively, in another embodiment, mutations can be introduced randomly along all or part of a 20750 coding sequence, such as by saturation mutagenesis, and the resultant mutants can be screened for 20750 biological activity to identify mutants that retain activity. Following mutagenesis of SEQ ID NO:1 the encoded protein can be expressed recombinantly and the activity of the protein can be determined using the assay described herein.

Another aspect of the invention pertains to the use of isolated nucleic acid molecules which are antisense to the nucleotide sequence of SEQ ID NO:1. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 20750 coding strand, or to only a portion thereof. In one embodiment, an antisense nucleic acid molecule is antisense to a “coding region” of the coding strand of a nucleotide sequence encoding a 20750. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding 20750. The term “noncoding region” refers to 5′ and 3′ sequences which flank the coding region that are not translated into amino acids (also referred to as 5′ and 3′ untranslated regions).

Given the coding strand sequences encoding 20750 disclosed herein, antisense nucleic acids of the invention can be designed according to the rules of Watson and Crick base pairing. The antisense nucleic acid molecule can be complementary to the entire coding region of 20750 mRNA, but more preferably is an oligonucleotide which is antisense to only a portion of the coding or noncoding region of 20750 mRNA. For example, the antisense oligonucleotide can be complementary to the region surrounding the translation start site of 20750 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyl uracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules used in the methods of the invention are typically administered to a subject or generated in situ such that they hybridize with or bind to cellular mRNA and/or genomic DNA encoding a 20750 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave 20750 mRNA transcripts to thereby inhibit translation of 20750 mRNA. A ribozyme having specificity for a 20750-encoding nucleic acid can be designed based upon the nucleotide sequence of a 20750 cDNA disclosed herein (i.e., SEQ ID NO:1). For example, a derivative of a Tetrahyniena L-19 IVS RNA can be constructed in which the nucleotide sequence of the active site is complementary to the nucleotide sequence to be cleaved in a 20750-encoding mRNA. See, e.g., Cech et al. U.S. Pat. No. 4,987,071; and Cech et al. U.S. Pat. No. 5,116,742. Alternatively, 20750 mRNA can be used to select a catalytic RNA having a specific ribonuclease activity from a pool of RNA molecules. See, e.g., Bartel, D. and Szostak, J. W. (1993) Science 261:1411-1418.

Alternatively, 20750 gene expression can be inhibited by targeting nucleotide sequences complementary to the regulatory region of the 20750 (e.g., the 20750 promoter and/or enhancers) to form triple helical structures that prevent transcription of the 20750 gene in target cells. See generally, Helene, C. (1991) Anticancer Drug Des. 6(6): 569-84; Helene, C. et al. (1992) Ann. N.Y. Acad. Sci. 660:27-36; and Maher, L. J. (1992) Bioassays 14(12):807-15.

In yet another embodiment, the 20750 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. (1996) Proc. Natl. Acad. Sci. 93:14670-675.

PNAs of 20750 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of 20750 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe et al. (1996) supra).

In another embodiment, PNAs of 20750 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 20750 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. et al. (1996) supra). The synthesis of PNA-DNA chimeras can be performed as described in Hyrup B. et al. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′DNA segment and a 3′PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

VI. Isolated 20750 Proteins and Anti-20750 Antibodies of the Invention

The methods of the invention include the use of isolated 20750 proteins, and biologically active portions thereof, as well as polypeptide fragments suitable for use as immunogens to raise anti-20750 antibodies. In one embodiment, native 20750 proteins can be isolated from cells or tissue sources by an appropriate purification scheme using standard protein purification techniques. In another embodiment, 20750 proteins are produced by recombinant DNA techniques. Alternative to recombinant expression, a 20750 protein or polypeptide can be synthesized chemically using standard peptide synthesis techniques.

As used herein, a “biologically active portion” of a 20750 protein includes a fragment of a 20750 protein having a 20750 activity. Biologically active portions of a 20750 protein include peptides comprising amino acid sequences sufficiently identical to or derived from the amino acid sequence of the 20750 protein, e.g., the amino acid sequence shown in SEQ ID NO:2, which include fewer amino acids than the full length 20750 proteins, and exhibit at least one activity of a 20750 protein. Typically, biologically active portions comprise a domain or motif with at least one activity of the 20750 protein. A biologically active portion of a 20750 protein can be a polypeptide which is, for example, 25, 50, 75, 100, 125, 150, 175, 200, 215, 250, 300, 350, 400 or more amino acids in length. Biologically active portions of a 20750 protein can be used as targets for developing agents which modulate a 20750 activity.

In a preferred embodiment, the 20750 protein used in the methods of the invention has an amino acid sequence shown in SEQ ID NO:2. In other embodiments, the 20750 protein is substantially identical to SEQ ID NO:2, and retains the functional activity of the protein of SEQ ID NO:2, yet differs in amino acid sequence due to natural allelic variation or mutagenesis, as described in detail in subsection V above. Accordingly, in another embodiment, the 20750 protein used in the methods of the invention is a protein which comprises an amino acid sequence at least about 50%, 55%, 59%, 60%, 65%, 70%, 75%, 80%, 85%, 90%, 95%, 96%, 97%, 98%, 99% or more identical to SEQ ID NO:2.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 20750 amino acid sequence of SEQ ID NO:2 having 439 amino acid residues, at least 75, preferably at least 150, more preferably at least 225, even more preferably at least 300, and even more preferably at least 400 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package (available online through the Genetics Computer Group), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available online through the Genetics Computer Group), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The methods of the invention may also use 20750 chimeric or fusion proteins. As used herein, a 20750 “chimeric protein” or “fusion protein” comprises a 20750 polypeptide operatively linked to a non-20750 polypeptide. An “20750 polypeptide” refers to a polypeptide having an amino acid sequence corresponding to a 20750 molecule, whereas a “non-20750 polypeptide” refers to a polypeptide having an amino acid sequence corresponding to a protein which is not substantially homologous to the 20750 protein, e.g., a protein which is different from the 20750 protein and which is derived from the same or a different organism. Within a 20750 fusion protein the 20750 polypeptide can correspond to all or a portion of a 20750 protein. In a preferred embodiment, a 20750 fusion protein comprises at least one biologically active portion of a 20750 protein. In another preferred embodiment, a 20750 fusion protein comprises at least two biologically active portions of a 20750 protein. Within the fusion protein, the term “operatively linked” is intended to indicate that the 20750 polypeptide and the non-20750 polypeptide are fused in-frame to each other. The non-20750 polypeptide can be fused to the N-terminus or C-terminus of the 20750 polypeptide.

For example, in one embodiment, the fusion protein is a GST-20750 fusion protein in which the 20750 sequences are fused to the C-terminus of the GST sequences. Such fusion proteins can facilitate the purification of recombinant 20750.

In another embodiment, this fusion protein is a 20750 protein containing a heterologous signal sequence at its N-terminus. In certain host cells (e.g., mammalian host cells), expression and/or secretion of 20750 can be increased through use of a heterologous signal sequence.

The 20750 fusion proteins used in the methods of the invention can be incorporated into pharmaceutical compositions and administered to a subject in vivo. The 20750 fusion proteins can be used to affect the bioavailability of a 20750 substrate. Use of 20750 fusion proteins may be useful therapeutically for the treatment of disorders caused by, for example, (i) aberrant modification or mutation of a gene encoding a 20750 protein; (ii) mis-regulation of the 20750 gene; and (iii) aberrant post-translational modification of a 20750 protein.

Moreover, the 20750-fusion proteins used in the methods of the invention can be used as immunogens to produce anti-20750 antibodies in a subject, to purify 20750 ligands and in screening assays to identify molecules which inhibit the interaction of 20750 with a 20750 substrate.

Preferably, a 20750 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A 20750-encoding nucleic acid can be cloned into such an expression vector such that the fusion moiety is linked in-frame to the 20750 protein.

The present invention also pertains to the use of variants of the 20750 proteins which function as either 20750 agonists (mimetics) or as 20750 antagonists. Variants of the 20750 proteins can be generated by mutagenesis, e.g., discrete point mutation or truncation of a 20750 protein. An agonist of the 20750 proteins can retain substantially the same, or a subset, of the biological activities of the naturally occurring form of a 20750 protein. An antagonist of a 20750 protein can inhibit one or more of the activities of the naturally occurring form of the 20750 protein by, for example, competitively modulating a 20750-mediated activity of a 20750 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 20750 protein.

In one embodiment, variants of a 20750 protein which function as either 20750 agonists (mimetics) or as 20750 antagonists can be identified by screening combinatorial libraries of mutants, e.g., truncation mutants, of a 20750 protein for 20750 protein agonist or antagonist activity. In one embodiment, a variegated library of 20750 variants is generated by combinatorial mutagenesis at the nucleic acid level and is encoded by a variegated gene library. A variegated library of 20750 variants can be produced by, for example, enzymatically ligating a mixture of synthetic oligonucleotides into gene sequences such that a degenerate set of potential 20750 sequences is expressible as individual polypeptides, or alternatively, as a set of larger fusion proteins (e.g., for phage display) containing the set of 20750 sequences therein. There are a variety of methods which can be used to produce libraries of potential 20750 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 20750 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a 20750 protein coding sequence can be used to generate a variegated population of 20750 fragments for screening and subsequent selection of variants of a 20750 protein. In one embodiment, a library of coding sequence fragments can be generated by treating a double stranded PCR fragment of a 20750 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the 20750 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 20750 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 20750 variants (Arkin and Yourvan (1992) Proc. Natl. Acad. Sci. USA 89:7811-7815; Delgrave et al. (1993) Protein Engineering 6(3):327-331).

The methods of the present invention further include the use of anti-20750 antibodies. An isolated 20750 protein, or a portion or fragment thereof, can be used as an immunogen to generate antibodies that bind 20750 using standard techniques for polyclonal and monoclonal antibody preparation. A full-length 20750 protein can be used or, alternatively, antigenic peptide fragments of 20750 can be used as immunogens. The antigenic peptide of 20750 comprises at least 8 amino acid residues of the amino acid sequence shown in SEQ ID NO:2 and encompasses an epitope of 20750 such that an antibody raised against the peptide forms a specific immune complex with the 20750 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of 20750 that are located on the surface of the protein, e.g., hydrophilic regions, as well as regions with high antigenicity.

A 20750 immunogen is typically used to prepare antibodies by immunizing a suitable subject, (e.g., rabbit, goat, mouse, or other mammal) with the immunogen. An appropriate immunogenic preparation can contain, for example, recombinantly expressed 20750 protein or a chemically synthesized 20750 polypeptide. The preparation can further include an adjuvant, such as Freund's complete or incomplete adjuvant, or similar immunostimulatory agent. Immunization of a suitable subject with an immunogenic 20750 preparation induces a polyclonal anti-20750 antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a 20750. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind 20750 molecules. The term “monoclonal antibody” or “monoclonal antibody composition”, as used herein, refers to a population of antibody molecules that contain only one species of an antigen binding site capable of immunoreacting with a particular epitope of 20750. A monoclonal antibody composition thus typically displays a single binding affinity for a particular 20750 protein with which it immunoreacts.

Polyclonal anti-20750 antibodies can be prepared as described above by immunizing a suitable subject with a 20750 immunogen. The anti-20750 antibody titer in the immunized subject can be monitored over time by standard techniques, such as with an enzyme linked immunosorbent assay (ELISA) using immobilized 20750. If desired, the antibody molecules directed against 20750 can be isolated from the mammal (e.g., from the blood) and further purified by well known techniques, such as protein A chromatography to obtain the IgG fraction. At an appropriate time after immunization, e.g., when the anti-20750 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somzatic Cell Genet. 3:231-36). Briefly, an immortal cell line (typically a mycloma) is fused to lymphocytes (typically splenocytes) from a mammal immunized with a 20750 immunogen as described above, and the culture supernatants of the resulting hybridoma cells are screened to identify a hybridoma producing a monoclonal antibody that binds 20750.

Any of the many well known protocols used for fusing lymphocytes and immortalized cell lines can be applied for the purpose of generating an anti-20750 monoclonal antibody (see, e.g., G. Galfre et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 20750, e.g., using a standard ELISA assay.

Alternative to preparing monoclonal antibody-secreting hybridomas, a monoclonal anti-20750 antibody can be identified and isolated by screening a recombinant combinatorial immunoglobulin library (e.g., an antibody phage display library) with 20750 to thereby isolate immunoglobulin library members that bind 20750. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. (1990) Nature 348:552-554.

Additionally, recombinant anti-20750 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-20750 antibody can be used to detect 20750 protein (e.g., in a cellular lysate or cell supernatant) in order to evaluate the abundance and pattern of expression of the 20750 protein. Anti-20750 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

VII. Electronic Apparatus Readable Media and Arrays

Electronic apparatus readable media comprising a 20750 modulator of the present invention is also provided. As used herein, “electronic apparatus readable media” refers to any suitable medium for storing, holding or containing data or information that can be read and accessed directly by an electronic apparatus. Such media can include, but are not limited to: magnetic storage media, such as floppy discs, hard disc storage medium, and magnetic tape; optical storage media such as compact disc; electronic storage media such as RAM, ROM, EPROM, EEPROM and the like; general hard disks and hybrids of these categories such as magnetic/optical storage media. The medium is adapted or configured for having recorded thereon a marker of the present invention.

As used herein, the term “electronic apparatus” is intended to include any suitable computing or processing apparatus or other device configured or adapted for storing data or information. Examples of electronic apparatus suitable for use with the present invention include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems.

As used herein, “recorded” refers to a process for storing or encoding information on the electronic apparatus readable medium. Those skilled in the art can readily adopt any of the presently known methods for recording information on known media to generate manufactures comprising the 20750 modulators of the present invention.

A variety of software programs and formats can be used to store the marker information of the present invention on the electronic apparatus readable medium. For example, the nucleic acid sequence corresponding to the 20750 modulators can be represented in a word processing text file, formatted in commercially-available software such as WordPerfect and MicroSoft Word, or represented in the form of an ASCII file, stored in a database application, such as DB2, Sybase, Oracle, or the like, as well as in other forms. Any number of dataprocessor structuring formats (e.g., text file or database) may be employed in order to obtain or create a medium having recorded thereon the 20750 modulators of the present invention.

By providing the 20750 modulators of the invention in readable form, one can routinely access the marker sequence information for a variety of purposes. For example, one skilled in the art can use the nucleotide or amino acid sequences of the present invention in readable form to compare a target sequence or target structural motif with the sequence information stored within the data storage means. Search means are used to identify fragments or regions of the sequences of the invention which match a particular target sequence or target motif.

The present invention therefore provides a medium for holding instructions for performing a method for determining whether a subject has a cellular proliferation disorder or a pre-disposition to a cellular proliferation disorder, wherein the method comprises the steps of determining the presence or absence of a 20750 modulator and based on the presence or absence of the 20750 modulator, determining whether the subject has a cellular proliferation disorder or a pre-disposition to cellular proliferation disorder and/or recommending a particular treatment for the cellular proliferation disorder or pre-cellular proliferation disorder condition.

The present invention further provides in an electronic system and/or in a network, a method for determining whether a subject has a cellular proliferation disorder or a pre-disposition to a cellular proliferation disorder associated with a 20750 modulator wherein the method comprises the steps of determining the presence or absence of the 20750 modulator, and based on the presence or absence of the 20750 modulator, determining whether the subject has a cellular proliferation disorder or a pre-disposition to a cellular proliferation disorder, and/or recommending a particular treatment for the cellular proliferation disorder or pre-cellular proliferation disorder condition. The method may further comprise the step of receiving phenotypic information associated with the subject and/or acquiring from a network phenotypic information associated with the subject.

The present invention also provides in a network, a method for determining whether a subject has a cellular proliferation disorder or a pre-disposition to a cellular proliferation disorder associated with a 20750 modulator, said method comprising the steps of receiving information associated with the 20750 modulator receiving phenotypic information associated with the subject, acquiring information from the network corresponding to the 20750 modulator and/or cellular proliferation disorder, and based on one or more of the phenotypic information, the 20750 modulator, and the acquired information, determining whether the subject has a cellular proliferation disorder or a pre-disposition to a cellular proliferation disorder. The method may further comprise the step of recommending a particular treatment for the cellular proliferation disorder or pre-cellular proliferation disorder condition.

The present invention also provides a business method for determining whether a subject has a cellular proliferation disorder or a pre-disposition to a cellular proliferation disorder, said method comprising the steps of receiving information associated with the 20750 modulator, receiving phenotypic information associated with the subject, acquiring information from the network corresponding to the 20750 modulator and/or cellular proliferation disorder, and based on one or more of the phenotypic information, the 20750 modulator, and the acquired information, determining whether the subject has a cellular proliferation disorder or a pre-disposition to a cellular proliferation disorder. The method may further comprise the step of recommending a particular treatment for the cellular proliferation disorder or pre-cellular proliferation disorder condition.

The invention also includes an array comprising a 20750 modulator of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of cellular proliferation disorder, progression of cellular proliferation disorder, and processes, such a cellular transformation associated with cellular proliferation disorder.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes that could serve as a molecular target for diagnosis or therapeutic intervention.

This invention is further illustrated by the following examples which should not be construed as limiting. The contents of all references, patents and published patent applications cited throughout this application and the Sequence Listing are incorporated herein by reference.

### EXAMPLES

### Example 1

### Identification and Characterization of Human 20750 cDNA

In this example, the identification and characterization of the gene encoding human 20750 is described.

Isolation of the Human 20750 cDNA

The invention is based, at least in part, on the discovery of a human gene encoding a novel polypeptide, referred to herein as human 20750. The entire sequence of the human clone 55053 was determined and found to contain an open reading frame termed human “20750.” The nucleotide sequence of the human 20750 gene is set forth in the Sequence Listing as SEQ ID NO:1. The amino acid sequence of the human 20750 expression product is set forth in the Sequence Listing as SEQ ID NO: 2. The 20750 polypeptide comprises about 439 amino acids. The coding region (open reading frame) of SEQ ID NO:1 is set forth as SEQ ID NO:3.

Analysis of the Human 20750 Molecules

A search using the polypeptide sequence of SEQ ID NO:2 was performed against the HMM database in PFAM resulting in the identification of a protein kinase domain in the amino acid sequence of human 20750 at about residues 1-201 of SEQ ID NO:2 (score=275.7).

A search using the polypeptide sequence of SEQ ID NO:2 was also performed against the Memsat database, resulting in the identification of a potential transmembrane domain (score=2.9) in the amino acid sequence of human 20750 (SEQ ID NO:2) at about residues 130-147.

Searches of the amino acid sequence of human 20750 were further performed against the Prosite database. These searches resulted in the identification in the amino acid sequence of human 20750 of five potential cAMP/cGMP-dependant protein kinase phosphorylation sites (ProSite Acc. No. PS00004) at about residues 188-191, 224-227, 260-263, 277-280, and 394-397 of SEQ ID NO:2. A glycosaminoglycan attachment site (ProSite Acc. No. PS00002) was also identified at about residues 350-353. Eleven potential protein kinase C phosphorylation sites (ProSite Ace. No. PS00005) were identified at about residues 18-20, 276-278, 284-286, 289-291, 305-307, 333-335, 375-377, 389-391, 393-395, 430-432, and 435-437 of SEQ ID NO:2. Two potential N-myristoylation sites (ProSite Acc. No. PS00008) were identified at about residues 237-242 and 349-354 of SEQ ID NO:2. Two amidation sites (ProSite Acc. No. PS00009) were identified at about residues 119-122 and 222-225 of SEQ ID NO:2. Three potential casein kinase II phosphorylation sites (ProSite Acc. No. PS00006) were identified at about residues 30-33, 101-104, and 172-175 of SEQ ID NO:2. A Serine/threonine protein kinase active site signature (ProSite Acc. No. PS00108) was identified at about residues 68-80 of SEQ ID NO:2.

The amino acid sequence of human 20750 was analyzed using the program PSORT to predict the localization of the proteins within the cell. This program assesses the presence of different targeting and localization amino acid sequences within the query sequence. The results of the analyses show that human 20750 may be localized to the cytoplasm, nucleus, or peroxisomes.

Further homologies of interest, including possible kinase-related domains, were identified by using the amino acid sequence of 20750 (SEQ ID NO:2) to search the ProDom database (available online through the ProDom (Protein Domain Database) website).

### Example 2

### Tissue Distribution of 20750 mRNA Using Taqman™ Analysis

This example describes the tissue distribution of human 20750 mRNA in a variety of cells and tissues, as determined using the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest, including, for example, lung, ovary, breast, and colon tumor samples, and normal samples, and used as the starting material for PCR amplification. In addition to the 5′ and 3′ gene-specific primers, a gene-specific oligonucleotide probe (complementary to the region being amplified) was included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-di methoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

A human panel comprising normal and tumorigenic tissues indicated broad distribution of human 20750 expression, with highest expression in normal brain cortex tissue and human umbilical vein endothelial cells (HUVEC) (see Table 1). Importantly, as shown in Table 1, human 20750 expression was increased 3-fold in colon tumor samples and lung tumor samples as compared to normal colon tissue samples and normal lung tissue samples.

A xenograft panel comprising breast, colon, lung and ovarian cancer cell lines as well as 293 and 293T cell lines was also tested. As shown in Table 2, expression of human 20750 was detected in cell lines of all origins, e.g., including colon, breast, and lung cancer cell lines.

An oncology human panel comprising normal and solid tumor samples indicated a 7-fold to 10-fold overexpression of human 20750 in breast, lung, and colon tumor samples as compared to normal breast, lung, and colon samples (see Table 3).

A panel comprising normal colon samples, early stage adenocarcinomas samples, colon to liver metastasis samples, and normal liver samples was also tested. As shown in Table 4, human 20750 is overexpressed in 95% of colon to liver metastasis samples.

An expanded colon panel comprising samples from various stages of colon cancer including stage B adenocarcinoma samples, stage C adenocarcinoma samples, adenoma samples, colon to liver metastasis samples, abdominal colon metastasis samples, normal colon samples, and normal liver samples was also tested (see Table 5). Results showed overexpression in colon to liver metastasis samples and significant expression in adenomas and stage A primary tumors.

An in vitro synchronized cell cycle panel was also tested (see Table 6). Expression of human 20750 was tested in several cell cycle regulated cancer cell lines at various time points. Abnormalities in cell cycle regulation and its checkpoints lead to the development of malignant cells. The loss of a cell's ability to respond to signals that regulate cell proliferation and cell cycle arrest is a common mechanism by which cancer develops. By synchronizing cell lines with drugs which cause cell cycle arrest, time points can be profiled to identify genes which are regulated in various stages of the cell cycle. Rapidly replicating human cells progress though the full cell cycle in about 24 hours (mitosis takes about 30 minutes, G1 takes about 9 hours, the S phase takes about 10 hours, and the G2 phase takes about 4.5 hours). Expression of human 20750 was tested at various time points in several cancer cell lines which were synchronized and induced to enter the cell cycle. Results show 20750 expression at all time points and increased 20750 expression in human adenocarcinoma DLD-1 cells.

A panel comprising cells from in vitro oncogene cell models was also tested. These oncogene cell models comprise cell lines transiently and stably transfected with tumor suppressors and oncogenes known to be associated with cancer progression, e.g., colon cancer progression. As shown in Tables 7 and 8, human 20750 is expressed in these various cancer cell models.

20750 expression was also analyzed in a Smad3^{-/-} mouse model. The Smad3^{-/-} mouse is a useful and unique model for human colorectal carcinogenesis. Smad3^{-/-} mice develop colon carcinomas that histopathologically resemble human disease. Samples from several stages of disease progression can be isolated, including normal epithelium, hyperplastic epithelium, adenomatous polyps, and various degrees of primary carcinoma and lymph node metastases. Expression of human 20750 in normal colon samples and adenoma samples at 12-14 weeks and 18-24 weeks was investigated. Results are shown in Table 9 and indicate that 20750 expression is upregulated in early and late stage tumors as compared to normal colon tissue.

Analysis of 20750 expression in cell cycle regulated HCT-116 human colon carcinoma cells treated with Nocodazole was also investigated. Nocodazole regulates the cell cycle in G2/M phase. Results show that human 20750 expression is upregulated during the G2/M phase of the cell cycle in these cells (see Table 10), indicating 20750 expression during cell proliferation.

The foregoing data reveal a significant up-regulation of 20750 mRNA in carcinomas, in particular colon carcinomas, colon metastases to the liver, breast carcinomas, and lung carcinomas. Moreover, these data link the expression of 20750 with cellular proliferation. Given that 20750 is expressed in a variety of tumors, with significant up-regulation in tumor samples as compared to normal samples, and that 20750 is expressed during cellular proliferation, it is believed that inhibition of 20750 activity may inhibit tumor formation or progression, especially in colon, breast, or lung tumors.

### Example 3

### Tissue Distribution of 20750 mRNA Using In Situ Analysis

For in situ analysis, various tissues, e.g., tissues obtained from normal colon, liver, breast, and lung and colon, breast, and lung tumors, and colon metastases to the liver were first frozen on dry ice. Ten-micrometer-thick sections of the tissues were post-fixed with 4% formaldehyde in DEPC treated 1× phosphate-buffered saline at room temperature for 10 minutes before being rinsed twice in DEPC 1× phosphate-buffered saline and once in 0.1 M triethanolamine-HCl (pH 8.0). Following incubation in 0.25% acetic anhydride-0.1 M triethanolamine-HCl for 10 minutes, sections were rinsed in DEPC 2× SSC (1× SSC is 0.15M NaCl plus 0.015M sodium citrate). Tissue was then dehydrated through a series of ethanol washes, incubated in 100% chloroform for 5 minutes, and then rinsed in 100% ethanol for 1 minute and 95% ethanol for 1 minute and allowed to air dry.

Hybridizations were performed with ³⁵S-radiolabeled (5×10⁷ cpm/ml) cRNA probes. Probes were incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1× Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides were washed with 2× SSC. Sections were then sequentially incubated at 37° C. in TNE (a solution containing 10 mM Tris-HCl (pH 7.6), 500 mM NaCl, and 1 mM EDTA), for 10 minutes, in TNE with 10 μg of RNase A per ml for 30 minutes, and finally in TNE for 10 minutes. Slides were then rinsed with 2× SSC at room temperature, washed with 2× SSC at 50° C. for 1 hour, washed with 0.2× SSC at 55° C. for 1 hour, and 0.2× SSC at 60° C. for 1 hour. Sections were then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

In situ hybridization results indicated expression of 20750 in one of four normal colon tissue samples tested, six of six colon tumor samples tested, five of five liver metastasis to the liver samples tested, and in two of two normal liver samples tested. Moderate to strong expression in primary colon tumor samples and metastatic carcinoma samples was also observed. Normal liver samples and normal colon samples show modest expression of 20750 as compared to colon tumor samples. Results further indicate expression of 20750 in two of two normal breast tissue samples tested and in four of four breast tumor tissue samples tested with modest expression in normal ductal epithelium as compared to breast tumor tissue. Results also indicate moderate to strong expression in four of four lung tumor tissues tested and two of two normal lung tissues tested.

These results, which confirm the expression pattern shown by Taqman analysis described above, indicate widespread expression of 20750 in most tumor types. 20750 is differentially expressed in colon tumors and liver metastases as compared to normal colon and liver tissue; in breast tumors as compared to normal breast tissue; and in lung tumors as compared to normal lung tissue. Therefore, inhibition of 20750 may inhibit tumor progression or formation, especially in colon tumors.

### Example 4

### Expression of Recombinant 20750 Protein in Bacterial Cells

In this example, human 20750 is expressed as a recombinant glutathione-S-transferase (GST) fusion polypeptide in E. coli and the fusion polypeptide is isolated and characterized. Specifically, 20750 is fused to GST and this fusion polypeptide is expressed in E. coli, e.g., strain PEB199. Expression of the GST-20750 fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 5

### Expression of Recombinent 20750 Protein in COS Cells

To express the human 20750 gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire 20750 protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the 20750 DNA sequence is amplified by PCR using two primers. The 5′ primer contains the restriction site of interest followed by approximately twenty nucleotides of the 20750 coding sequence starting from the initiation codon; the 3′ end sequence contains complementary sequences to the other restriction site of interest, a translation stop codon, the HA tag or FLAG tag and the last 20 nucleotides of the 20750 coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the 20750 gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the 20750-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the 20750 polypeptide is detected by radiolabelling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA-specific monoclonal antibody. Precipitated polypeptides are then analyzed by SDS-PAGE.

Alternatively, DNA containing the 20750 coding sequence is cloned directly into the polylinker of the pCDNA/Amp vector using the appropriate restriction sites. The resulting plasmid is transfected into COS cells in the manner described above, and the expression of the 20750 polypeptide is detected by radiolabelling and immunoprecipitation using an 20750 specific monoclonal antibody.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying a compound capable of treating a cell proliferation disorder, comprising assaying the ability of the compound to modulate 20750 nucleic acid expression or 20750 polypeptide activity, thereby identifying a compound capable of treating a cell proliferation disorder.

2. A method for identifying a compound capable of modulating cellular proliferation comprising:
a) contacting a cell which expresses 20750 with a test compound; and
b) assaying the ability of the test compound to modulate the expression of a 20750 nucleic acid or the activity of a 20750 polypeptide, thereby identifying a compound capable of modulating cellular proliferation.

3. A method for modulating cellular proliferation in a cell comprising contacting a cell with a 20750 modulator, thereby modulating cellular proliferation in the cell.

4. The method of claim 2, wherein the cell is a breast cell, a lung cell or a colon cell.

5. The method of claim 3, wherein the 20750 modulator is a small organic molecule, peptide, antibody or antisense nucleic acid molecule.

6. The method of claim 3, wherein the 20750 modulator is capable of modulating 20750 polypeptide activity.

7. The method of claim 6, wherein the 20750 modulator is a small organic molecule peptide, antibody or antisense nucleic acid molecule.

8. The method of claim 6, wherein the 20750 modulator is capable of modulating 20750 nucleic acid expression.

9. A method for treating a subject having a cell proliferation disorder characterized by aberrant 20750 polypeptide activity or aberrant 20750 nucleic acid expression comprising administering to the subject a 20750 modulator, thereby treating said subject having a cell proliferation disorder.

10. The method of claim 9, wherein said cell proliferation disorder is selected from the group consisting of breast cancer, lung cancer and colon cancer.

11. The method of claim 9, wherein said 20750 modulator is administered in a pharmaceutically acceptable formulation.

12. The method of claim 9, wherein the 20750 modulator is a small organic molecule, peptide, antibody or antisense nucleic acid molecule.

13. The method of claim 9, wherein the 20750 modulator is capable of modulating 20750 polypeptide activity.
